SEQUENCE LISTING (21 sequences)

SEQ ID NO: 1 - DNA, 3115 bp, Artificial Sequence
  Coq-3 knockout mutation
atgatcccttcacgaagtgccagaatcatcgcaaagctacaacgactacactcgactacttcagccgcttcagtatcttctattgatgtaaaagaggtaaaacatataaaaataagctatttatctgtagaaaaattattttaggtcgaaaaattcggagacttgtctgcagaatgggctgatgaactgggtcccttccacgcacttcactcattaaacaggattcgagttccttggattgtcgataatgttagaaaaagcgatcagaaggctcctcctcgattagtggacgttggaagcggagggggtcttttgtcgattccactggccagaagtggattcgatgttacaggaattgatgcgacgaagcaagctgtaagggagattttcccatttttctgggaatttatgcaaaatcagctctaagacatcaaaaactatgaaaatttatcggttttctcactgaaatattgtcattttttcaatttctttgattgaaattgcgttttaaattaccaaaaacgatctgatttttaaattttgcaaaaagcaaaatgccgcacagaaaagaggcggggcgatttggcaaccctgcggcacggttttttcttctgttattttcgcaaaaatcgccaattttacacagttttttgcaataaaattttgatttcacttgttttattcactttctattaaatattgtgtgaatatttcatgttttgcaaccaattttgcataaaatgttctcaaaatccaacatttcagtgagaaaatcgataaattttaatgttttggattaaaatagagctgatcttgcctaattatactgggtttaaatgaataatttccaggtagaagctgcgaatcagtccctcacagcgaaaccccttcaaattgccggaatctcgaagcgcctccggttcgagcataccagcgtcgaggatttctgtcagaagccacacaataaatcgggtacatttcttcttcctataggaacatttcattgttatcagggagataatttcgcttgtcagctgtcacatgagatttatctcctagaatttggaaaaaaatgttactcagaggccaggaatgcagaataatccccatttagtgaagtgtttcacaatgtttgcacttcgattttcaacatattttgacagctgcatttttcctaaaagactctgttaattgcatgacttcttttccgtctctccgtctctctgctgctgctctgctggttgacgtcttcttcagaagcttcaagcgccaaactatcgattttgaagagcccccgacaagtttttttcacagaaaaagtgctaaatatttcaataaagccggttttcggttttcacccgggggtaatcggaaggattactaccccattataccttgtagtgaagaatagttgtttgtaatggaggaattggatgggtattgttcagtgtactgtacagcgccagcagtggcttattgcagtctgtaaaagttataaaagtagtcctagaagcccccaagtttgggcaggaatttccgcattctctcaaaacatctcaattaatcttcctcctcgcgcactacacaccatcttcacagttgacttgaaattgagtcttctcgacgaatttcctttcttttttgttgaaaaaagtgttgatccaacccaattcaattcgatttccggtgcccccttggaataattttggatacaaagctttcaactcttctgttctgttctctatttccctattttgctcgccgtcttctcctcctccacccgtccggcttctcctcttcttggacattttatcgattttgttcttcttcggtgttgtgtctctctctctctccccccccccttttcgatgtgtgggccaacacaacaatccccacatttctgcgtctcgtgttctcaccctcatccggttgtgtctgcgtctatggcttgtaggttctcgaactttcagttctagatgtcctagacttcaattttgaaggtctcaactggatattattacagttcggaagtcttgaataatactagatccaacccagatgtcctcagatgttatttgatctctccagtctctcgccgtcgctcccttctctcagtccattttggacgctcatttcgaccgccatcccgtttggggttaaccgcggagagagtgagtgagaaagggaatgagcgctcaaattcactctcactcacactcacacgcagcagcatcatctcgtagaccctctctggttgttgctgtctctgatgacaaacattccctaactgggcgcccctgtgttcgtcgttgccacgtgtcattctatgtcggcgattcggccatttgaagctcgatccacgtgtcgctaggacagctgacgtcatcttttcaactattatgtttactgcgattatacgaatcaattggtgaaattatttagaataacctattttttgagttgtttacgattttgaagtcacttgactgaaaactttcacagaaaaggtcttaaatgaaatgaaactcttgcgtagacttgatgaagttctgtgaaactcctacgtactcttgaatagtaatcgaaaattattgatttctacttccaatctactcaaaagttaaaaaatatttcgcaacacatcttttccccattcttttctgtattttttagcaatttaccttaaaatcttcaataattccagcctacgatgcagtcgtcgcttcggaaattgtcgaacacgtcgccgatcttcccggattcattggctgcctcgctgagctggctcgccccggtgccccgctcttcatcacaactatcaacagaacgtggctgagcaaattggcagctatttggcttgcagaggtttgatttttttctttcttttttttttggaaataaatttgaaaattttcagaatgtactcaaaatcgtgccgcccggagtccacgactgggaaaaattcatcacacccgccgagctcacttcacatctcgaaaaagcgggttgccgggtgacggcggtgcatggattaatgtttcatccggttggaaatcactggacatggatcgaatcgactcagtgtaattacggaattttggcagtgaagaattag

SEQ ID NO: 2 - protein, 268 aa, Artificial Sequence
  methyltransferase
MIPSRSARIIAKLQRLHSTTSAASVSSIDVKEVEKFGDLSAEWADELGPFHALHSLNRIRVPWIVDNVRKSDQKAPPRLVDVGSGGGLLSIPLARSGFDVTGIDATKQAVEAANQSLTAKPLQIAGISKRLRFEHTSVEDFCQKPHNKSAYDAVVASEIVEHVADLPGFIGCLAELARPGAPLFITTINRTWLSKLAAIWLAENVLKIVPPGVHDWEKFITPAELTSHLEKAGCRVTAVHGLMFHPVGNHWTWIESTQCNYGILAVKN

SEQ ID NO: 3 - protein, 267 aa, Artificial Sequence
  Coq-3 proteins from C. elegans
MIPSRSARIIAKLQRLHSTTSAASVSSIDVKEVEKFGDLSAEWADELGPFHALHSLNRIRVPWIVDNVRKSDQKAPPLVDVGSGGGLLSIPLARSGFDVTGIDATKQAVEAANQSLTAKPLQIAGISKRLRFEHTSVEDFCQKPHNKSAYDAVVASEIVEHVADLPGFIGCLAELARPGAPLFITTINRTWLSKLAAIWLAENVLKIVPPGVHDWEKFITPAELTSHLEKAGCRVTAVHGLMFHPVGNHWTWIESTQCNYGILAVKN

SEQ ID NO: 4 - protein, 316 aa, Artificial Sequence
  Coq-3 proteins from S. cerevisiae
MGFIMLLRSRFLKVIHVRKQLSACSRFAIQTQTRCKSTDASEDEVKHFQELAPTWWDTDGSQRILHKMNLTRLDFVQRTVRNQVKIQNPEIFVPGFNYKEFLPEYVCDNIQREMQESIETNLDKRPEVSVLDVGCGGGILSESLARLKWVKNVQGIDLTRDCIMVAKEHAKKDPMLEGKINYECKALEDVTGQFDIITCMEMLEHVDMPSEILRHCWSRLNPEKGILFLSTINRDLISWFTTIFMGENVLKIVPKGTHHLSKYINSKEILAWFNDNYSGQFRLLDLKGTMYLPYQGWVEHDCSDVGNYFMAIQRLN

SEQ ID NO: 5 - protein, 240 aa, Artificial Sequence
  Coq-3 proteins from E. coli
MNAEKSPVNHNVDHEEIAKFEAVASRWWDLEGEFKPLHRINPLRLGYIAERAGGLFGKKVLDVGCGGGILAESMAREGATVTGLDMGFEPLQVAKLHALESGIQVDYVQETVEEHAAKHAGQYDVVTCMEMLEHVPDPQSVVRACAQLVKPGGDVFFSTLNRNGKSWLMAVVGAEYILRMVPKGTHDVKKFIKPAELLGWVDQTSLKERHITGLHYNPITNTFKLGPGVDVNYMLHTQNK

SEQ ID NO: 6 - protein, 249 aa, Artificial Sequence
  Coq-3 proteins from H. sapiens
MNDLRVPFIRDNLLKTIPNHQPGKLLGMKILDVGCGGGLLTEPLGRLGASVIGIDPVDENIKTAQCHKSFDPVLDKRIEYRVCSLEEIVEETAETFDAVVASEVVEHVIDLETFLQCCCQVLKPGGSLFITTINKTQLSYALGIVFSEQIASIVPKGTHTWEKFVSPETLESILESNGLSVQTVVGMLYNPFSGYWHWSENTSLNYAAYAVKSRVQEHPASAEFVLKGETEELQANACTNPAVHEKLKK

SEQ ID NO: 7 - DNA, 660 bp, Artificial Sequence
  2456 bp deletion in coq-3 (qm188)
atgatcccttcacgaagtgccagaatcatcgcaaagctacaacgactacactcgactacttcagccgcttcagtatcttctattgatgtaaaagaggtaaaacatataaaaataagctatttatctgtagaaaaattattttaggtcgaaaaattcggagacttgtctgcagaatgggctgatgaactgggtcccttccacgcacttcactcattaaacaggattcgagttccttggattgtcgataatgttagaaaaagcgatcagaaggctcctcctcgattagtggacgttggaagcggagggggtcttttgtcgattccactggccagaagtggattcgatgttacaggaattgatgcgacgaagcaagctgtaagggagattttcccatttttctgggaatttatgcaaaatcagctcttttcttttttttttggaaataaatttgaaaattttcagaatgtactcaaaatcgtgccgcccggagtccacgactgggaaaaattcatcacacccgccgagctcacttcacatctcgaaaaagcgggttgccgggtgacggcggtgcatggattaatgtttcatccggttggaaatcactggacatggatcgaatcgactcagtgtaattacggaattttggcagtgaagaattag

SEQ ID NO: 8 - DNA, 807 bp, Artificial Sequence
  Exons 4 of coq-3 (qm188)
atgatcccttcacgaagtgccagaatcatcgcaaagctacaacgactacactcgactacttcagccgcttcagtatcttctattgatgtaaaagaggtcgaaaaattcggagacttgtctgcagaatgggctgatgaactgggtcccttccacgcacttcactcattaaacaggattcgagttccttggattgtcgataatgttagaaaaagcgatcagaaggctcctcctcgattagtggacgttggaagcggagggggtcttttgtcgattccactggccagaagtggattcgatgttacaggaattgatgcgacgaagcaagctgtagaagctgcgaatcagtccctcacagcgaaaccccttcaaattgccggaatctcgaagcgcctccggttcgagcataccagcgtcgaggatttctgtcagaagccacacaataaatcggcctacgatgcagtcgtcgcttcggaaattgtcgaacacgtcgccgatcttcccggattcattggctgcctcgctgagctggctcgccccggtgccccgctcttcatcacaactatcaacagaacgtggctgagcaaattggcagctatttggcttgcagagaatgtactcaaaatcgtgccgcccggagtccacgactgggaaaaattcatcacacccgccgagctcacttcacatctcgaaaaagcgggttgccgggtgacggcggtgcatggattaatgtttcatccggttggaaatcactggacatggatcgaatcgactcagtgtaattacggaattttggcagtgaagaattag

SEQ ID NO: 9 - DNA, 30 bp, Artificial Sequence
  SHP 1772
ctgatttcttccagagctctcttgccgcac

SEQ ID NO: 10 - DNA, 30 bp, Artificial Sequence
  SHP 1773
agcattcccgagatgatgcactccttgagg

SEQ ID NO: 11 - DNA, 27 bp, Artificial Sequence
  SHP 1774
tagcgactctcagcgacaagcttaacc

SEQ ID NO: 12 - DNA, 27 bp, Artificial Sequence
  SHP 1775
gaggccggttccgagacgatggcatcg

SEQ ID NO: 13 - DNA, 24 bp, Artificial Sequence
  SHP 1840
cctcctcgcgcactacacaccatc

SEQ ID NO: 14 - DNA, 24 bp, Artificial Sequence
  SHP 1865
cgaagcgacgactgcatcgtaggc

SEQ ID NO: 15 - DNA, 10597 bp, Artificial Sequence
  Mus musculus wild-type mclk-1 locus (1)...(60): n = any
nttaggntcccggcngggggtttcgggggnattcaaacccaggttctttaacagggngcagggaatgtttttcaacttctgagctctctctagctctaattttttaaacactttacttccaaaaatttccagtaataactaggacatacacctcaacattcttttatctctttaggacagatctagaagtggaatgcacgggaaaggttctgaacatttgaaggctttgagagccagatttaggctgagtattccacaggaatatttaagtaccctcactgccgctaaggcccagtacttgggctcgtcttaattttaagttactgtagtatactaacttgaacactataattatatagaatgggttagtagtttcatttattttacagtagctatgaatcaaatacatacccccccgccaagggtgcaaactctagttttcctaaagccacagtctctagtgatcctataataggcactgattatgccttgcaggtatggtttttccccttatatacttatctgacttggaaattttatttctattggctagatctagctgtcatattaatggatatgcactttaaaatgtttaatgtatgctactaaattttccttccctcaacacacagctatgttcatcactaatgtgccctaggccatgaataatccagtaaaggatgaacaatgaagcaaatctcaatataaaaattgagaaggaacggaaagtaacgggaaacaaactgggaaacccacaattacaccgaaaactggcatgttttaccaggtaattgctgactttcaggtgtaaatcggctcttaatagagaaaaataatctccgtaacgtggaggaaacagtgacctgcgggtcgcctttccaagacagggagaaaccctaacctaaactgcctctccaggacagctctgcatcaaccctaagagccgaccgggcgccttcactacgttccaatgatcgacagggggcagaccaaatagagtacgtgaattggtcatttctaaccaatcgggtttaatccagggcctaggggcgtttcctctggctctcggtccgcggcatctatgcgtcatcaccctgagtcgagagcacgattggcggggcgtttggaccatagctgcattgtccgcagcgatgagcgccgccggagccatagcggctgcttccgtgggacgcctgcgcactggtgtccggaggcccttctcaggtaccggccgctcggggtcgtggttcggcgcggggttcttcgctggtgactatttgcagtggaggtcacggatgtcacggggaggacgtctatacgtcacaagcgcgcgacatgggggtggggtttgtagtacgtctagttgattgacaggaatgggtgaacttctgcaggatgccctgccgggggaacaagtgattaccagcctgtgatgtgacgtcagtgcaaggcacatcacagtgcaacttgagtgtcctgcagtgtccctccggtctccactcgggactttctcaagcaaagtagccttccgacgacagcatcaatctgttataaatgcagattttcgggtccccctcattatccactggattgataggttttagagttttaaaaagtgtgtgtgtgtgtgtgtgtgtgtgtgtgtgtgtgtgtgtgtgtgtgcgcgcgcgcgtgcgtgcgcacaataattaaattctagagaggccagaattggggtttgaatctgcaactagagtaacaggcagtttgtgaatccctatatatgggtgctgggagcccaactcaagtcatttgcaagtgcagtacacattcttaactgctgagccttcttttgagccgtagagtctctgtttttatcaagcctctcatgggactgtttgtattgcaaaatatggtaagctttgaatttggaatggagagaggtaggtttggattcaggtctgattcatccgagctgccccagaaaatcgtggtcattttcttgaaactaaatcttcaagcattagatttctattttgcctgaaggcaatattgtttggaggtttgagatgtgtttttgtttaactacaacttattaagtaatttaattgaaactacatccttttggtaaataataagccagaattgcctgcccccaaatggatgagtaatcaccccccccacacacacacaccaccaccaccaccaccaccaccaccaataccttgagaacagctatgaaacttcattagatattgttgtgtgtgccttccaaggcttgagtccaaggctgtttttctttctggaagagaagartgcttagcaagtgtttggatattattcaagacatgccagttatagagatgtttcccttggtgataatgattcagaagagactttttaagagcctcttagtatgtaatttatgtgtccataagccacttaaaatcttctcatttgccacttaaaatctcccagataccatggctggaacagcacgcttgagagcttctagctctgcgattcagctttattttaaagtgtgtaacaaggcagtcaggtctcagggatgtggattttaaggttccttgctaacccagtgaacaggttaacaataagaatcacttgttttcatttatgaagtaacttagtttcctttgtttcatataccctcatgataataataataataaaaaacccttaagtgtgtgtctccttaaaaaaataaaactagccttgctactgggtaggttttaatttggttttgagacagcctctagcgctggctgacctggaccttgcttgttcaccaggatatccttcaattcacagagctctgccaactcatgctgggattaatacctatactgatttcttaggtaaaaggacaaatactaccctctcccagtcattgagtgcagaagttgtgttttagagaacattccagtgtgttctcagttataaagaatcctgtttatcacacctcaaaagccagccatataaacttggcccacctgctcagctccttgttattcttcttttaaaaaaaagatttattttatatatgtgaacacactgtagctgtcttcagacacaccagaagagggaatcagatcccattacaaatggtcctgagccaccatgtagttgctgggaattgacctcaggacctctggaagagcagtcagtgcttttaaccgctgagccacctccccaggccccttgttattccttaatatactttttaaaaaggagtactgggttgccttgaactttctttgctaaaaagtaagagcacaagcaaacaagattgtgttgagaaatacaactggctcaccaagtctgtctccagaccctgctttctgcaaggagacaagctggccagaagcataagcccttgaacttggacacagaaatgcaacaagttcctgattgtgtcccatcactgtcccataaaatatgggcctcaaaccgtagagccccactgtctgaagacagtttggaatggttggtgtcttacactcggcaggagagctgaggtgccgtgtctgctccacagactctgtccagttagagtcagtgagctgagtgaggcagagcatgccatgcgcagtagaccaaagccattctccctcctgcagaatccacgtgcctttgcacacacagcgctatttgtcccaggactgttgatgtagctcagcatttaaaattctacttggtagcaaagctcacgttcccaacgactgcatgcatacacaccagatactccaatcctgccccgtggccttgtgtccaaagaccttaagccttggttgatgaaagagccaaagacatatgggacttttccacccgttttctggatgttgaagtttgcttaggtgaaaagaagtgtcttccaaagacatggtggtcatagcaagcagagagcctgcagcactttaaacagggtgcagctagagtgacaaaccagagggcctgtgggtttccgtttttatatggaataaacacacattactacaggacccttctgggatgaggtaaacattcaagatcctctaatctggagcttggaagtatagtgaagtgtttacatttgaagaagagtttagtctgaggtcaaaccttgtcaggcagggtctcagtcacctgcccgtggaattggtgtattaaaagaacgttgaagccccaacttgggatgccaggctttgtcccctgagccttttcagaacatcaacactggccgcttcccagggagacttagggagagcattatagatagctttgggtgcactccaggggcttctgtacagcttgagaggggagcctccctttcctgaaacagctgtcacgtcagctgccttgtgaggacagatttcggtccttccagatcgccatatgtttaaagtaaatccggaagccctagtctttaggtgaagtcttttgggtttgagcattgcaggtgacaaagaacacacactggtagatgtgtccagccctcaggcttgtctttcattctgtcggcaaaaaggcaacaggccagcgatatgactcagtgggtaaaggtgcttgctttccagcacaagggcctgagttccatccctggaccccacaactccgttttcaggaatgtcagtgtcctgtgtggataatgagacggacacttgctttttcattgcagagtatggaagaggcctcatcatcaggtgtcacagttcggggatgaccttagacaatattaaccgggcagccgtggatckaataattcgggtggatcacgctggtgaatatggagcaaaccgcatctatgcagggcaaatggctgtgctcggtcggaccagtgttggccctgtcattcaggtgggttctttcctgagtctcagcccagtctgttgccctggcagtgtatctgaagccctcgggcatcacttttggctgtgtgctccaaagggaggcacttggaacaaagcacttgctctgttgtctaaaagcacagatatgcattgactctggctgggtgtggtggtgcatgcctataatcccagcacttgggagctggagatagggtgatcgctgggactttgaggccagcctggtctacataggaagttccaggtcagaaagaaaaaaatggagagagggggaaagaaagtaagagagaaagaaattgggtctggaaattgggtgtatttgtggtgttaatgtttcattgcagaaaaggctgaaagtccctccattagaagaatgttccatgtgccaggaggttgttgtaggcttgtcctagcacagagtatcagagagaggggttaacagccccgaagatctaggtttcctttccagatctctcatctacttctgcgaccctgaagaggtcacctgacctctaggttttcatttccctgtgtgcacactagcctggtaacccccacctccctgggtctggctggggaataaaccagatcctgttgtcaccatgacacatggcagcttagatccccgcagatcccagtccccagtgctcatcccatgtgtaagatggtgggtgtctgcttgtggccctgcacaactctcctgtgaagagtccttcatgccaggagaatgcctctcattggctgtcctgttttctattgagaacattctgcgagttttcaggacacagttttgttgttgttgttgttgttagtttttttcattattttctcttgtggttgcttgagccggtggctcagaacctggagttctatatggctcactatgcaagctgattgtgtggtcactgaggtgtgtgtggctctggaggtggaacacttagctctgtccaaggccttggttcttcatttacttggcaggtgcttttcttttttgagagattcttctgtggtttgcttttatctcatggatatttaaggggatggaagacagcattgcaccaattccttcttacctcttgtgtgctcagcgagccgtgtccctgtgatgcctctttttatgtttccccccccagaaaatgtgggatcaagagaagaaccatttgaaaaagttcaacgagttgatgattgcattcagggtccgacctacggttttgatgcccttgtggaacgtggcaggctttgccctgggtatgtgtctgtccagcagccgcttgggctctaatgatgggctgttcctgcctctggagcccttgtcagggctgcatccaaccttttaaaatttactgtgtgttttcctaaagctaaattgaagttgatgaagttgatkgaattttctttgtttatattactttaagatagagccatcacttttataaatagatggtataataactcacagagggaagctaggatcgtgccaccactgccagaatccatgtcctgaggatcctgacctcagagcaacctgactgtgagagtgctggtgcccacctttaaccccagcactcgggagacagaggcaggcagatctctgagtttgaggccagcatggtctacaaatcgagttccataacacacacacacacacacacacacacacacacacacacacacacacacagaagaacagcagagaacccagatagcactctcagctctctgcagagggtcaagtctcattgagcccatgtgttaacttgggtttcatagtgagatcttgtctcaaacaaaacaaaccaaccaaataaaataaaaatccattcagaaagagctttgtgactggcatctgatataagctccagccgcttctcaactaggcgtgactgtttcaagggattcatgggaatatctgaatgcccagtggtcatgatcagcaggtactgctgacatccagagggtggatatcgggtgccattagacaccctgagaaacacgtcacagccctcccagagagttaccaacccaggtgtcaggacgcctcacagatgaccagcagcctgtggcttgactttgtttgtttgacggttgcaggggcaggaactgccttgctggggaaggaaggagcaatggcctgcaccgtggcggtagaagagtctatcgctaatcactacaacaaccagatccgcatgctgatggaagaggaccctgagaagtatgaggagctgctgcaggtgatgactgtgcgctgcttgaggagagaaagggcaggtgacaggagatgggtactaaggaggcagggacttagacagctggggaagggggcgtatcttttacgtgagacacagacagatcatacagctcagaactgttcccagtccaggtctgtgtggcctctgcacatccatgactcagcagcacgaggtgaacaaggatgatgtcagctaacacactaactagacagagaaaaatccacaaggcctgacccctacacaaagaaccatagtgatgcaggaaggtcgagatgggaggggtggccttctgtttgtccagtgccagaaggtcagcctgaaagcatacatacaggtggcattatgcggacagaagagactagatttaaatatgtataagcaaatacatacacacaggcaacagcaactaatgaaaagagaagccatgaacttgaaggagagcagagaggggtatatgggaggaaggaaagggacaggaaaaaatgctgtggttaactaataatcccaaaaataaaataaaaaaaatgatgatcaactcttcaggttgagtgattttcctcaggtttctctatagaaaagaaggaactatttggccctgggctggtcttaaaactagcgtctacagaggtcctcctgcctggttgccatcctccagcactctccctaacagcagttcatttacttagattctgtttggtttacttttgagacaaaggcttgtcttgactcttggccctcctgcctctgccttccaagggctggggatgtcagtgtgtattgctgtacttggccatgtggtggtttgaataagcgcaggcccccacagtttcacatatctgaatgcttagatgtgggggagtggcattatttgagaagggttaggaggctcaggattagccttgttggaggaagtatgttgttggagggtggggctttaagcccatgccaggcccagggtctgtctcttggtctgcaagtcaggatgtagctctcggctactgctccagcaccaaagtgctgccctgctccctgctaagctgatagtgagctaaacctctgaaacctcaggcaagcccccagttaaatgctttcctttctaagagttgctttcctcatggtgtctcttcacagcaacagagcagggactaagacaggcaacaactctcactttttaaaacctaaagtcagccactggctgaccctagcctgtggccatgctcgtttcgtaaataagtctcattagagccacagctatgggttactcttgcaaggctgttcaccccactggagtgccagggtagaaaaagcatgagagcctttgacagctgtatgtgaggacacaggctctggcctggaaacaggatgagctgccggcaacctggggtgccgactcaccccagtctgcgattcctttcttcccaggtcatcaagcagtttcgcgatgaggagcttgaacaccacgacacaggcctggaccacgatgcagagctggtagggccaactcttcttgtgctgctctcgggccattttaaaggttgtgggggacaaaggtttctgttcccaaaaggagacatttgaaagtacaggtcagaaggcagggaaacgggtacttgacagaaagcacccaagctcagccttggtccatggtgaggctcctgtgtcctgctctgttactaacacaagaaacaacccagcagttcagtgtccatagatgcttctagaatttcaaatggcttttgtttcaaattaaatcatttcccaratcctctttttatccagaggagcccaaaccctgccctaccagtgagtccaggtctgaacatctgaaaatagatgcatctcgtgggggtttccttgctgtttgtttaggggctggcattgaatccagggccttgctaggcaagcgctctaccacttaacagaccacttgcccgtttgcttattttcccagctcagggtgccgccgtgcatgttagacaatactctaccatctagtacatcgcagccttttgttctccgcaggctcccgcgtatgccttgttgaagaggattatccaggccggatgcagtgcagccatatatttatcagaaaggttttagagtatgtctattgatccatttctagaaaagatggtcgtaacttaaggagtgatgtttgtggaggaggtgctgtacagttatcactgtgtgtgttttgttaatacaaaaggccgggtttggggcttgtgtttgtcaataaactctttggcgctggattccttggttttcttgtgctgtgaggttggcagttaactaactctgctcaccttacagtacctgcagctggtcttcccttggtcttatagttaatttgggcctaagacatcaagaacaaaccattcgtcagttaacaggaatccttttttaaagatttattttacttctatttctagagtttaaaaacattagactgtataagatgggctaagcaagactgggaagtctctcgagggaggtgctgtgcattctgatgtcagcatgatgccgcaaagcactgtggtagctatggctcctgaaaatcctcacccagagtcgatggtaggaggtggtaaatccctcaccccagaggagacacctgaagggagaggaggctgggaggtggcagataaggggcagagacctcaggagtggggttagtgcccttatagaaacgaggcctagggagacccagtctgttccacatcactggacaccaacctgttggcaccctgatattggacttcatagcctccagaactgcaaacaagtttttgttgttcatgagctcctgagcctacagtattttaatagcagtcctggcagactaaggcaggatggcattatcccaatcaaaaatatacttaagttgggtgtggtgatgcaggcctgtaatcctagcaccatgggaggcagaggcaagaagatctgcaggagtcccagggctatcctcagcacacgtcaagtttgaggacagcgtacatgacacccggccccagcaaacaaccacaataacatacagagctgtgggttatttacaattgaattataatttctgcaaggtctgctatctccaaataagccagactgacaaaaatttagtatttctgtgaactattttattattttaaattttcaaaatatatttaaagaaaaacaaacaaacaaacaaagaacccaggatcaagcagagtgtggtgatacatgcctgtaatcccagccgtgggagcagagggagagagatcttcatgagccagttggttacgtagcaagaccctgtcaaatacaaaagccaaaaaaaaaaaaaaaaaaacctcagttctcctcagaatgtcctttcaaacttccctgggaggctgaggcaggagttaaaggtcagtctgagcaatacmgcaagaaaaaaaaacmaatgaatttgcagaccaaaatctgacctagttgcactggtcagtggtccctatagcgarcctgagatgactggggctt

SEQ ID NO: 16 - DNA, 9353 bp, Artificial Sequence
  Mus musculus mutant knockout allele of mclk-1 (1)...(60): n = any
nttaggntcccggcngggggtttcgggggnattcaaacccaggttctttaacagggngcagggaatgtttttcaacttctgagctctctctagctctaattttttaaacactttacttccaaaaatttccagtaataactaggacatacacctcaacattcttttatctctttaggacagatctagaagtggaatgcacgggaaaggttctgaacatttgaaggctttgagagccagatttaggctgagtattccacaggaatatttaagtaccctcactgccgctaaggcccagtacttgggctcgtcttaattttaagttactgtagtatactaacttgaacactataattatatagaatgggttagtagtttcatttattttacagtagctatgaatcaaatacatacccccccgccaagggtgcaaactctagttttcctaaagccacagtctctagtgatcctataataggcactgattatgccttgcaggtatggtttttccccttatatacttatctgacttggaaattttatttctattggctagatctagctgtcatattaatggatatgcactttaaaatgtttaatgtatgctactaaattttccttccctcaacacacagctatgttcatcactaatgtgccctaggccatgaataatccagtaaaggatgaacaatgaagcaaatctcaatataaaaattgagaaggaacggaaagtaacgggaaacaaactgggaaacccacaattacaccgaaaactggcatgttttaccaggtaattgctgactttcaggtgtaaatcggctcttaatagagaaaaataatctccgtaacgtggaggaaacagtgacctgcgggtcgcctttccaagacagggagaaaccctaacctaaactgcctctccaggacagctctgcatcaaccctaagagccgaccgggcgccttcactacgttccaatgatcgacagggggcagaccaaatagagtacgtgaattggtcatttctaaccaatcgggtttaatccagggcctaggggcgtttcctctggctctcggtccgcggcatctatgcgtcatcaccctgagtcgagagcacgattggcggggcgtttggaccatagctgcattgtccgcagcgatgagcgccgccggagccatagcggctgcttccgtgggacgcctgcgcactggtgtccggaggcccttctcaggtaccggccgctcggggtcgtggttcggcgcggggttcttcgctggtgactatttgcagtggaggtcacggatgtcacggggaggacgtctatacgtcacaagcgcgcgacatgggggtggggtttgtagtacgtctagttgattgacaggaatgggtgaacttctgcaggatgccctgccgggggaacaagtgattaccagcctgtgatgtgacgtcagtgcaaggcacatcacagtgcaacttgagtgtcctgcagtgtccctccggtctccactcgggactttctcaagcaaagtagccttccgacgacagcatcaatctgttataaatgcagattttcgggtccccctcattatccactggattgataggttttagagttttaaaaagtgtgtgtgtgtgtgtgtgtgtgtgtgtgtgtgtgtgtgtgtgtgtgcgcgcgcgcgtgcgtgcgcacaataattaaattctagagaggccagaattggggtttgaatctgcaactagagtaacaggcagtttgtgaatccctatatatgggtgctgggagcccaactcaagtcatttgcaagtgcagtacacattcttaactgctgagccttcttttgagccgtagagtctctgtttttatcaagcctctcatgggactgtttgtattgcaaaatatggtaagctttgaatttggaatggagagaggtaggtttggattcaggtctgattcatccgagctgccccagaaaatcgtggtcattttcttgaaactaaatcttcaagcattagatttctattttgcctgaaggcaatattgtttggaggtttgagatgtgtttttgtttaactacaacttattaagtaatttaattgaaactacatccttttggtaaataataagccagaattgcctgcccccaaatggatgagtaatcaccccccccacacacacacaccaccaccaccaccaccaccaccaccaataccttgagaacagctatgaaacttcattagatattgttgtgtgtgccttccaaggcttgagtccaaggctgtttttctttctggaagagaagartgcttagcaagtgtttggatattattcaagacatgccagttatagagatgtttcccttggtgataatgattcagaagagactttttaagagcctcttagtatgtaatttatgtgtccataagccacttaaaatcttctcatttgccacttaaaatctcccagataccatggctggaacagcacgcttgagagcttctagctctgcgattcagctttattttaaagtgtgtaacaaggcagtcaggtctcagggatgtggattttaaggttccttgctaacccagtgaacaggttaacaataagaatcacttgttttcatttatgaagtaacttagtttcctttgtttcatataccctcatgataataataataataaaaaacccttaagtgtgtgtctccttaaaaaaataaaactagccttgctactgggtaggttttaatttggttttgagacagcctctagcgctggctgacctggaccttgcttgttcaccaggatatccttcaattcacagagctctgccaactcatgctgggattaatacctatactgatttcttaggtaaaaggacaaatactaccctctcccagtcattgagtgcagaagttgtgttttagagaacattccagtgtgttctcagttataaagaatcctgtttatcacacctcaaaagccagccatataaacttggcccacctgctcagctccttgttattcttcttttaaaaaaaagatttattttatatatgtgaacacactgtagctgtcttcagacacaccagaagagggaatcagatcccattacaaatggtcctgagccaccatgtagttgctgggaattgacctcaggacctctggaagagcagtcagtgcttttaaccgctgagccacctccccaggccccttgttattccttaatatactttttaaaaaggagtactgggttgccttgaactttctttgctaaaaagtaagagcacaagcaaacaagattgtgttgagaaatacaactggctcaccaagtctgtctccagaccctgctttctgcaaggagacaagctggccagaagcataagcccttgaacttggacacagaaatgcaacaagttcctgattgtgtcccatcactgtcccataaaatatgggcctcaaaccgtagagccccactgtctgaagacagtttggaatggttggtgtcttacactcggcaggagagctgaggtgccgtgtctgctccacagactctgtccagttagagtcagtgagctgagtgaggcagagcatgccatgcgcagtagaccaaagccattctccctcctgcagaatccacgtgcctttgcacacacagcgctatttgtcccaggactgttgatgtagctcagcatttaaaattctacttggtagcaaagctcacgttcccaacgactgcatgcatacacaccagatactccaatcctgccccgtggccttgtgtccaaagaccttaagccttggttgatgaaagagccaaagacatatgggacttttccacccgttttctggatgttgaagtttgcttaggtgaaaagaagtgtcttccaaagacatggtggtcatagcaagcagagagcctgcagcactttaaacagggtgcagctagagtgacaaaccagagggcctgtgggtttccgtttttatatggaataaacacacattactacaggacccttctgggatgaggtaaacattcaagatcctctaatctggagcttggaagtatagtgaagtgtttacatttgaagaagagtttagtctgaggtcaaaccttgtcaggcagggtctcagtcacctgcccgtggaattggtgtattaaaagaacgttgaagccccaacttgggatgccaggctttgtcccctgagccttttcagaacatcaacactggccgcttcccagggagacttagggagagcattatagatagctttgggtgcactccaggggcttctgtacagcttgagaggggagcctccctttcctgaaacagctgtcacgtcagctgccttgtgaggacagatttcggtccttccagatcgccatatgtttaaagtaaatccggaagccctagtctttaggtgaagtcttttgggtttgagcattgcaggtgacaaagaacacacactggtagatgtgtccagccctcaggcttgtctttcattctgtcggcaaaaaggcaacaggccagcgatatgactcagtgggtaaaggtgcttgctttccagcacaagggcctgagttccatccctggaccccacaactccgttttcaggaatgtcagtgtcctgtgtggataatgagacggacacttgctttttcattgcagagtatggaagaggctcgagcagtgtggttttgcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatcccccatcgaattcctgcaggtgatgactgtgcgctgcttgaggagagaaagggcaggtgacaggagatgggtactaaggaggcagggacttagacagctggggaagggggcgtatcttttacgtgagacacagacagatcatacagctcagaactgttcccagtccaggtctgtgtggcctctgcacatccatgactcagcagcacgaggtgaacaaggatgatgtcagctaacacactaactagacagagaaaaatccacaaggcctgacccctacacaaagaaccatagtgatgcaggaaggtcgagatgggaggggtggccttctgtttgtccagtgccagaaggtcagcctgaaagcatacatacaggtggcattatgcggacagaagagactagatttaaatatgtataagcaaatacatacacacaggcaacagcaactaatgaaaagagaagccatgaacttgaaggagagcagagaggggtatatgggaggaaggaaagggacaggaaaaaatgctgtggttaactaataatcccaaaaataaaataaaaaaaatgatgatcaactcttcaggttgagtgattttcctcaggtttctctatagaaaagaaggaactatttggccctgggctggtcttaaaactagcgtctacagaggtcctcctgcctggttgccatcctccagcactctccctaacagcagttcatttacttagattctgtttggtttacttttgagacaaaggcttgtcttgactcttggccctcctgcctctgccttccaagggctggggatgtcagtgtgtattgctgtacttggccatgtggtggtttgaataagcgcaggcccccacagtttcacatatctgaatgcttagatgtgggggagtggcattatttgagaagggttaggaggctcaggattagccttgttggaggaagtatgttgttggagggtggggctttaagcccatgccaggcccagggtctgtctcttggtctgcaagtcaggatgtagctctcggctactgctccagcaccaaagtgctgccctgctccctgctaagctgatagtgagctaaacctctgaaacctcaggcaagcccccagttaaatgctttcctttctaagagttgctttcctcatggtgtctcttcacagcaacagagcagggactaagacaggcaacaactctcactttttaaaacctaaagtcagccactggctgaccctagcctgtggccatgctcgtttcgtaaataagtctcattagagccacagctatgggttactcttgcaaggctgttcaccccactggagtgccagggtagaaaaagcatgagagcctttgacagctgtatgtgaggacacaggctctggcctggaaacaggatgagctgccggcaacctggggtgccgactcaccccagtctgcgattcctttcttcccaggtcatcaagcagtttcgcgatgaggagcttgaacaccacgacacaggcctggaccacgatgcagagctggtagggccaactcttcttgtgctgctctcgggccattttaaaggttgtgggggacaaaggtttctgttcccaaaaggagacatttgaaagtacaggtcagaaggcagggaaacgggtacttgacagaaagcacccaagctcagccttggtccatggtgaggctcctgtgtcctgctctgttactaacacaagaaacaacccagcagttcagtgtccatagatgcttctagaatttcaaatggcttttgtttcaaattaaatcatttcccaratcctctttttatccagaggagcccaaaccctgccctaccagtgagtccaggtctgaacatctgaaaatagatgcatctcgtgggggtttccttgctgtttgtttaggggctggcattgaatccagggccttgctaggcaagcgctctaccacttaacagaccacttgcccgtttgcttattttcccagctcagggtgccgccgtgcatgttagacaatactctaccatctagtacatcgcagccttttgttctccgcaggctcccgcgtatgccttgttgaagaggattatccaggccggatgcagtgcagccatatatttatcagaaaggttttagagtatgtctattgatccatttctagaaaagatggtcgtaacttaaggagtgatgtttgtggaggaggtgctgtacagttatcactgtgtgtgttttgttaatacaaaaggccgggtttggggcttgtgtttgtcaataaactctttggcgctggattccttggttttcttgtgctgtgaggttggcagttaactaactctgctcaccttacagtacctgcagctggtcttcccttggtcttatagttaatttgggcctaagacatcaagaacaaaccattcgtcagttaacaggaatccttttttaaagatttattttacttctatttctagagtttaaaaacattagactgtataagatgggctaagcaagactgggaagtctctcgagggaggtgctgtgcattctgatgtcagcatgatgccgcaaagcactgtggtagctatggctcctgaaaatcctcacccagagtcgatggtaggaggtggtaaatccctcaccccagaggagacacctgaagggagaggaggctgggaggtggcagataaggggcagagacctcaggagtggggttagtgcccttatagaaacgaggcctagggagacccagtctgttccacatcactggacaccaacctgttggcaccctgatattggacttcatagcctccagaactgcaaacaagtttttgttgttcatgagctcctgagcctacagtattttaatagcagtcctggcagactaaggcaggatggcattatcccaatcaaaaatatacttaagttgggtgtggtgatgcaggcctgtaatcctagcaccatgggaggcagaggcaagaagatctgcaggagtcccagggctatcctcagcacacgtcaagtttgaggacagcgtacatgacacccggccccagcaaacaaccacaataacatacagagctgtgggttatttacaattgaattataatttctgcaaggtctgctatctccaaataagccagactgacaaaaatttagtatttctgtgaactattttattattttaaattttcaaaatatatttaaagaaaaacaaacaaacaaacaaagaacccaggatcaagcagagtgtggtgatacatgcctgtaatcccagccgtgggagcagagggagagagatcttcatgagccagttggttacgtagcaagaccctgtcaaatacaaaagccaaaaaaaaaaaaaaaaaaacctcagttctcctcagaatgtcctttcaaacttccctgggaggctgaggcaggagttaaaggtcagtctgagcaatacmgcaagaaaaaaaaacmaatgaatttgcagaccaaaatctgacctagttgcactggtcagtggtccctatagcgarcctgagatgactggggctt

SEQ ID NO: 17 - DNA, 25 bp, Artificial Sequence
  KO5
ggtgaagtcttttgggtttgagcat

SEQ ID NO: 18 - DNA, 25 bp, Artificial Sequence
  KO6
tgtctaaggtcatccccgaactgtg

SEQ ID NO: 19 - DNA, 25 bp, Artificial Sequence
  KO7
gccagcgatatgactcagtgggtaa

SEQ ID NO: 20 - DNA, 25 bp, Artificial Sequence
  KO8
caccttaatatgcgaagtggacctg

SEQ ID NO: 21 - DNA, 10853 bp, Artificial Sequence
  mclk-1 flox allele (1)...(60): n = any
nttaggntcccggcngggggtttcgggggnattcaaacccaggttctttaacagggngcagggaatgtttttcaacttctgagctctctctagctctaattttttaaacactttacttccaaaaatttccagtaataactaggacatacacctcaacattcttttatctctttaggacagatctagaagtggaatgcacgggaaaggttctgaacatttgaaggctttgagagccagatttaggctgagtattccacaggaatatttaagtaccctcactgccgctaaggcccagtacttgggctcgtcttaattttaagttactgtagtatactaacttgaacactataattatatagaatgggttagtagtttcatttattttacagtagctatgaatcaaatacatacccccccgccaagggtgcaaactctagttttcctaaagccacagtctctagtgatcctataataggcactgattatgccttgcaggtatggtttttccccttatatacttatctgacttggaaattttatttctattggctagatctagctgtcatattaatggatatgcactttaaaatgtttaatgtatgctactaaattttccttccctcaacacacagctatgttcatcactaatgtgccctaggccatgaataatccagtaaaggatgaacaatgaagcaaatctcaatataaaaattgagaaggaacggaaagtaacgggaaacaaactgggaaacccacaattacaccgaaaactggcatgttttaccaggtaattgctgactttcaggtgtaaatcggctcttaatagagaaaaataatctccgtaacgtggaggaaacagtgacctgcgggtcgcctttccaagacagggagaaaccctaacctaaactgcctctccaggacagctctgcatcaaccctaagagccgaccgggcgccttcactacgttccaatgatcgacagggggcagaccaaatagagtacgtgaattggtcatttctaaccaatcgggtttaatccagggcctaggggcgtttcctctggctctcggtccgcggcatctatgcgtcatcaccctgagtcgagagcacgattggcggggcgtttggaccatagctgcattgtccgcagcgatgagcgccgccggagccatagcggctgcttccgtgggacgcctgcgcactggtgtccggaggcccttctcaggtaccggccgctcggggtcgtggttcggcgcggggttcttcgctggtgactatttgcagtggaggtcacggatgtcacggggaggacgtctatacgtcacaagcgcgcgacatgggggtggggtttgtagtacgtctagttgattgacaggaatgggtgaacttctgcaggatgccctgccgggggaacaagtgattaccagcctgtgatgtgacgtcagtgcaaggcacatcacagtgcaacttgagtgtcctgcagtgtccctccggtctccactcgggactttctcaagcaaagtagccttccgacgacagcatcaatctgttataaatgcagattttcgggtccccctcattatccactggattgataggttttagagttttaaaaagtgtgtgtgtgtgtgtgtgtgtgtgtgtgtgtgtgtgtgtgtgtgtgcgcgcgcgcgtgcgtgcgcacaataattaaattctagagaggccagaattggggtttgaatctgcaactagagtaacaggcagtttgtgaatccctatatatgggtgctgggagcccaactcaagtcatttgcaagtgcagtacacattcttaactgctgagccttcttttgagccgtagagtctctgtttttatcaagcctctcatgggactgtttgtattgcaaaatatggtaagctttgaatttggaatggagagaggtaggtttggattcaggtctgattcatccgagctgccccagaaaatcgtggtcattttcttgaaactaaatcttcaagcattagatttctattttgcctgaaggcaatattgtttggaggtttgagatgtgtttttgtttaactacaacttattaagtaatttaattgaaactacatccttttggtaaataataagccagaattgcctgcccccaaatggatgagtaatcaccccccccacacacacacaccaccaccaccaccaccaccaccaccaataccttgagaacagctatgaaacttcattagatattgttgtgtgtgccttccaaggcttgagtccaaggctgtttttctttctggaagagaagartgcttagcaagtgtttggatattattcaagacatgccagttatagagatgtttcccttggtgataatgattcagaagagactttttaagagcctcttagtatgtaatttatgtgtccataagccacttaaaatcttctcatttgccacttaaaatctcccagataccatggctggaacagcacgcttgagagcttctagctctgcgattcagctttattttaaagtgtgtaacaaggcagtcaggtctcagggatgtggattttaaggttccttgctaacccagtgaacaggttaacaataagaatcacttgttttcatttatgaagtaacttagtttcctttgtttcatataccctcatgataataataataataaaaaacccttaagtgtgtgtctccttaaaaaaataaaactagccttgctactgggtaggttttaatttggttttgagacagcctctagcgctggctgacctggaccttgcttgttcaccaggatatccttcaattcacagagctctgccaactcatgctgggattaatacctatactgatttcttaggtaaaaggacaaatactaccctctcccagtcattgagtgcagaagttgtgttttagagaacattccagtgtgttctcagttataaagaatcctgtttatcacacctcaaaagccagccatataaacttggcccacctgctcagctccttgttattcttcttttaaaaaaaagatttattttatatatgtgaacacactgtagctgtcttcagacacaccagaagagggaatcagatcccattacaaatggtcctgagccaccatgtagttgctgggaattgacctcaggacctctggaagagcagtcagtgcttttaaccgctgagccacctccccaggccccttgttattccttaatatactttttaaaaaggagtactgggttgccttgaactttctttgctaaaaagtaagagcacaagcaaacaagattgtgttgagaaatacaactggctcaccaagtctgtctccagaccctgctttctgcaaggagacaagctggccagaagcataagcccttgaacttggacacagaaatgcaacaagttcctgattgtgtcccatcactgtcccataaaatatgggcctcaaaccgtagagccccactgtctgaagacagtttggaatggttggtgtcttacactcggcaggagagctgaggtgccgtgtctgctccacagactctgtccagttagagtcagtgagctgagtgaggcagagcatgccatgcgcagtagaccaaagccattctccctcctgcagaatccacgtgcctttgcacacacagcgctatttgtcccaggactgttgatgtagctcagcatttaaaattctacttggtagcaaagctcacgttcccaacgactgcatgcatacacaccagatactccaatcctgccccgtggccttgtgtccaaagaccttaagccttggttgatgaaagagccaaagacatatgggacttttccacccgttttctggatgttgaagtttgcttaggtgaaaagaagtgtcttccaaagacatggtggtcatagcaagcagagagcctgcagcactttaaacagggtgcagctagagtgacaaaccagagggcctgtgggtttccgtttttatatggaataaacacacattactacaggacccttctgggatgaggtaaacattcaagatcctctaatctggagcttggaagtatagtgaagtgtttacatttgaagaagagtttagtctgaggtcaaaccttgtcaggcagggtctcagtcacctgcccgtggaattggtgtattaaaagaacgttgaagccccaacttgggatgccaggctttgtcccctgagccttttcagaacatcaacactggccgcttcccagggagacttagggagagcattatagatagctttgggtgcactccaggggcttctgtacagcttgagaggggagcctccctttcctgaaacagctgtcacgtcagctgccttgtgaggacagatttcggtccttccagatcgccatatgtttaaagtaaatccggagctagcgagctcggaataacttcgtataatgtatgctatacgaagttatggcgaattccggaagccctagtctttaggtgaagtcttttgggtttgagcattgcaggtgacaaagaacacacactggtagatgtgtccagccctcaggcttgtctttcattctgtcggcaaaaaggcaacaggccagcgatatgactcagtgggtaaaggtgcttgctttccagcacaagggcctgagttccatccctggaccccacaactccgttttcaggaatgtcagtgtcctgtgtggataatgagacggacacttgctttttcattgcagagtatggaagaggcctcatcatcaggtgtcacagttcggggatgaccttagacaatattaaccgggcagccgtggatckaataattcgggtggatcacgctggtgaatatggagcaaaccgcatctatgcagggcaaatggctgtgctcggtcggaccagtgttggccctgtcattcaggtgggttctttcctgagtctcagcccagtctgttgccctggcagtgtatctgaagccctcgggcatcacttttggctgtgtgctccaaagggaggcacttggaacaaagcacttgctctgttgtctaaaagcacagatatgcattgactctggctgggtgtggtggtgcatgcctataatcccagcacttgggagctggagatagggtgatcgctgggactttgaggccagcctggtctacataggaagttccaggtcagaaagaaaaaaatggagagagggggaaagaaagtaagagagaaagaaattgggtctggaaattgggtgtatttgtggtgttaatgtttcattgcagaaaaggctgaaagtccctccattagaagaatgttccatgtgccaggaggttgttgtaggcttgtcctagcacagagtatcagagagaggggttaacagccccgaagatctaggtttcctttccagatctctcatctacttctgcgaccctgaagaggtcacctgacctctaggttttcatttccctgtgtgcacactagcctggtaacccccacctccctgggtctggctggggaataaaccagatcctgttgtcaccatgacacatggcagcttagatccccgcagatcccagtccccagtgctcatcccatgtgtaagatggtgggtgtctgcttgtggccctgcacaactctcctgtgaagagtccttcatgccaggagaatgcctctcattggctgtcctgttttctattgagaacattctgcgagttttcaggacacagttttgttgttgttgttgttgttagtttttttcattattttctcttgtggttgcttgagccggtggctcagaacctggagttctatatggctcactatgcaagctgattgtgtggtcactgaggtgtgtgtggctctggaggtggaacacttagctctgtccaaggccttggttcttcatttacttggcaggtgcttttcttttttgagagattcttctgtggtttgcttttatctcatggatatttaaggggatggaagacagcattgcaccaattccttcttacctcttgtgtgctcagcgagccgtgtccctgtgatgcctctttttatgtttccccccccagaaaatgtgggatcaagagaagaaccatttgaaaaagttcaacgagttgatgattgcattcagggtccgacctacggttttgatgcccttgtggaacgtggcaggctttgccctgggtatgtgtctgtccagcagccgcttgggctctaatgatgggctgttcctgcctctggagcccttgtcagggctgcatccaaccttttaaaatttactgtgtgttttcctaaagctaaattgaagttgatgaagttgatkgaattttctttgtttatattactttaagatagagccatcacttttataaatagatggtataataactcacagagggaagctaggatcgtgccaccactgccagaatccatgtcctgaggatcctgacctcagagcaacctgactgtgagagtgctggtgcccacctttaaccccagcactcgggagacagaggcaggcagatctctgagtttgaggccagcatggtctacaaatcgagttccataacacacacacacacacacacacacacacacacacacacacacacacacagaagaacagcagagaacccagatagcactctcagctctctgcagagggtcaagtctcattgagcccatgtgttaacttgggtttcatagtgagatcttgtctcaaacaaaacaaaccaaccaaataaaataaaaatccattcagaaagagctttgtgactggcatctgatataagctccagccgcttctcaactaggcgtgactgtttcaagggattcatgggaatatctgaatgcccagtggtcatgatcagcaggtactgctgacatccagagggtggatatcgggtgccattagacaccctgagaaacacgtcacagccctcccagagagttaccaacccaggtgtcaggacgcctcacagatgaccagcagcctgtggcttgactttgtttgtttgacggttgcaggggcaggaactgccttgctggggaaggaaggagcaatggcctgcaccgtggcggtagaagagtctatcgctaatcactacaacaaccagatccgcatgctgatggaagaggaccctgagaagtatgaggagctgctgcaggtgatgactgtgcgctgcttgaggagagaaagggcaggtgacaggagatgggtactaaggaggcagggacttagacagctggggaagggggcgtatcttttacgtgagacacagacagatcatacagctcagaactgttcccagtccaggtctgtgtggcctctgcacatccatgactcagcagcacgaggtgaacaaggatgatgtcagctaacacactaactagacagagaaaaatccacaaggcctgacccctacacaaagaaccatagtgatgcaggaaggtcgagatgggaggggtggccttctgtttgtccagtgccagaaggtcagcctgaaagcatacatacaggtggcattatgcggacagaagagactagattttcgaggtcgacgcatgcctgtacatccggagacgcgtcacggccgaagctagcgaattccgatcatattcaataacccttaatataacttcgtataatgtatgctatacgaagttattaggtctgaagaggagtttacgtccagccaagctagcttggctgcagcccgggggatccactagttctagagcggccaaatatgtataagcaaatacatacacacaggcaacagcaactaatgaaaagagaagccatgaacttgaaggagagcagagaggggtatatgggaggaaggaaagggacaggaaaaaatgctgtggttaactaataatcccaaaaataaaataaaaaaaatgatgatcaactcttcaggttgagtgattttcctcaggtttctctatagaaaagaaggaactatttggccctgggctggtcttaaaactagcgtctacagaggtcctcctgcctggttgccatcctccagcactctccctaacagcagttcatttacttagattctgtttggtttacttttgagacaaaggcttgtcttgactcttggccctcctgcctctgccttccaagggctggggatgtcagtgtgtattgctgtacttggccatgtggtggtttgaataagcgcaggcccccacagtttcacatatctgaatgcttagatgtgggggagtggcattatttgagaagggttaggaggctcaggattagccttgttggaggaagtatgttgttggagggtggggctttaagcccatgccaggcccagggtctgtctcttggtctgcaagtcaggatgtagctctcggctactgctccagcaccaaagtgctgccctgctccctgctaagctgatagtgagctaaacctctgaaacctcaggcaagcccccagttaaatgctttcctttctaagagttgctttcctcatggtgtctcttcacagcaacagagcagggactaagacaggcaacaactctcactttttaaaacctaaagtcagccactggctgaccctagcctgtggccatgctcgtttcgtaaataagtctcattagagccacagctatgggttactcttgcaaggctgttcaccccactggagtgccagggtagaaaaagcatgagagcctttgacagctgtatgtgaggacacaggctctggcctggaaacaggatgagctgccggcaacctggggtgccgactcaccccagtctgcgattcctttcttcccaggtcatcaagcagtttcgcgatgaggagcttgaacaccacgacacaggcctggaccacgatgcagagctggtagggccaactcttcttgtgctgctctcgggccattttaaaggttgtgggggacaaaggtttctgttcccaaaaggagacatttgaaagtacaggtcagaaggcagggaaacgggtacttgacagaaagcacccaagctcagccttggtccatggtgaggctcctgtgtcctgctctgttactaacacaagaaacaacccagcagttcagtgtccatagatgcttctagaatttcaaatggcttttgtttcaaattaaatcatttcccaratcctctttttatccagaggagcccaaaccctgccctaccagtgagtccaggtctgaacatctgaaaatagatgcatctcgtgggggtttccttgctgtttgtttaggggctggcattgaatccagggccttgctaggcaagcgctctaccacttaacagaccacttgcccgtttgcttattttcccagctcagggtgccgccgtgcatgttagacaatactctaccatctagtacatcgcagccttttgttctccgcaggctcccgcgtatgccttgttgaagaggattatccaggccggatgcagtgcagccatatatttatcagaaaggttttagagtatgtctattgatccatttctagaaaagatggtcgtaacttaaggagtgatgtttgtggaggaggtgctgtacagttatcactgtgtgtgttttgttaatacaaaaggccgggtttggggcttgtgtttgtcaataaactctttggcgctggattccttggttttcttgtgctgtgaggttggcagttaactaactctgctcaccttacagtacctgcagctggtcttcccttggtcttatagttaatttgggcctaagacatcaagaacaaaccattcgtcagttaacaggaatccttttttaaagatttattttacttctatttctagagtttaaaaacattagactgtataagatgggctaagcaagactgggaagtctctcgagggaggtgctgtgcattctgatgtcagcatgatgccgcaaagcactgtggtagctatggctcctgaaaatcctcacccagagtcgatggtaggaggtggtaaatccctcaccccagaggagacacctgaagggagaggaggctgggaggtggcagataaggggcagagacctcaggagtggggttagtgcccttatagaaacgaggcctagggagacccagtctgttccacatcactggacaccaacctgttggcaccctgatattggacttcatagcctccagaactgcaaacaagtttttgttgttcatgagctcctgagcctacagtattttaatagcagtcctggcagactaaggcaggatggcattatcccaatcaaaaatatacttaagttgggtgtggtgatgcaggcctgtaatcctagcaccatgggaggcagaggcaagaagatctgcaggagtcccagggctatcctcagcacacgtcaagtttgaggacagcgtacatgacacccggccccagcaaacaaccacaataacatacagagctgtgggttatttacaattgaattataatttctgcaaggtctgctatctccaaataagccagactgacaaaaatttagtatttctgtgaactattttattattttaaattttcaaaatatatttaaagaaaaacaaacaaacaaacaaagaacccaggatcaagcagagtgtggtgatacatgcctgtaatcccagccgtgggagcagagggagagagatcttcatgagccagttggttacgtagcaagaccctgtcaaatacaaaagccaaaaaaaaaaaaaaaaaaacctcagttctcctcagaatgtcctttcaaacttccctgggaggctgaggcaggagttaaaggtcagtctgagcaatacmgcaagaaaaaaaaacmaatgaatttgcagaccaaaatctgacctagttgcactggtcagtggtccctatagcgarcctgagatgactggggctt

(19) United States
(12) Patent Application Publication
(10) Pub. No.: US 2005/0039221 A1
(43) Pub. Date: Feb. 17, 2005

(54) Phenotypic effects of ubiquinone deficiencies and methods of screening thereof

(76) Inventors: Siegfried Hekimi, Montreal Quebec (CA); Abdelmadjid Hihi, Montreal (CA); Francoise Levavasseur, Gavray (FR); Eric Shoubridge, Westmont Quebec (CA); Yuan Gao, Danbury, CT (US)

Correspondence Address: JONES DAY, 222 EAST 41ST ST, NEW YORK, NY, 10017, US

(21) Appl. No.: 10/486,309

(22) PCT Filed: Aug. 7, 2002
(86) PCT No.: PCT/CA02/01230

Related U.S. Application Data
(60) Provisional application No. 60/310,231, filed on Aug. 7, 2001.

(51) Int. Cl. A01K 67/027; C12N 5/06

(52) U.S. Cl. 800/18; 435/354

(57) ABSTRACT

The present invention relates to a method of screening for a compound allowing survival of clk1 homozygous mutant embryos; a method of screening for a compound suitable for rescue of mutant phenotype of mclk1 homozygous cell line; a method of screening for a compound suitable for partial or complete functional replacement of endogenous ubiquinone; a method for screening a compound capable of inhibiting activity of clk-1 and/or other processes required to make ubiquinone from demethoxyubiquinone; a non-ubiquinone-producer mouse; a DNA construct, which comprises an alteration of mclk1; a non-ubiquinone-producer ES cell line; a coq-3 mutant subject non-ubiquinone producer, a method of screening for a compound suitable for complete or partial functional ubiquinone or demethoxyubiquinone replacement; a method for reducing and/or increasing ubiquinone level in a multicellular subject; a method of screening for a genetic suppressor of clk-1; and a method of screening for a genetic suppressor of coq-3.

### BACKGROUND OF THE INVENTION

(a) Field of the Invention

This invention relates to the phenotypic effects of ubiquinone deficiencies and methods of screening thereof.

(b) Description of Prior Art

Ubiquinone (UQ), and its reduced form ubiquinol, is a prenylated benzoquinone/ol lipid and is the major site of production of reactive oxygen species (ROS). It is a co-factor in the mitochondrial respiratory chain where it becomes reduced by the activity of Complex I and Complex II, and oxidized by the activity of Complex III. During these processes, ubisemiquinone species are formed, which are unstable and generate superoxide. Furthermore, ubiquinone/ubiquinol is a redox-active cofactor of other enzyme systems that produce ROS, for example the plasma membrane NAD(P)H oxidoreductases, as well as the lysosomal and peroxisomal electron transport chains. In all these locations ROS can be produced during redox reactions involving ubiquinone/ubiquinol.

In addition, ubiquinone is a ubiquitous natural anti-oxidant, whose presence in biological membranes helps to detoxify ROS produced by endogenous processes or by toxicants or radiations. Unfortunately, dietary ubiquinone has very poor penetration into cells, in particular into subcellular organelles.

Reactive oxygen species have been implicated in numerous human diseases, including, but not exclusively, diabetes (Nishikawa et al., (2000). Nature, 404, 787-790; Brownlee (2001). Nature 414, 813-820), hypoxia/reoxygenation injury (Li et al., (2002). Am J Physiol Cell Physiol 282, C227-C241; Lesnefsy et al., (2001). J. Mol Cell Cardiol 33, 1065-1089; Cuzzocrea et al., (2001). Pharmacological Reviews 53, 1, 135-159), Parkinson's (Betarbet et al., (2002). Bioessays 24, 308-318), atherosclerosis atherosclerosis (Lusis, (2000). Nature, 407, 233-241), and Alzheimer's disease (Butterfield et al., (2001). Trends in Molecular Medicine, 7, 12, 548-554; Tabner et al., (2002) Free Radical Biology & Medicine, 32, 11, 1076-1083, 2002).

The gene clk-1 of the nematode Caenorhabditis elegans affects many physiological rates, including embryonic and post-embryonic development, rhythmic behaviors, reproduction and life span. clk-1 encodes a 187 amino acid protein that localizes to mitochondria, and that is homologous to the yeast protein Coq7p, which has been shown to be required for UQ biosynthesis. clk-1 has also been shown to be necessary for UQ biosynthesis (Jonassen, T. et al., (2001). Proc Natl Acad Sci USA 98, 421-6.; Miyadera, H. et al., (2001). J. Biol Chem 276, 7713-6), as UQ₉ is entirely absent from mitochondria purified from clk-1 mutants (Miyadera, H. et al., (2001). J. Biol Chem 276, 7713-6) (the subscript refers to the length of the isoprenoid side chain). Instead, these mitochondria accumulate demethoxyubiquinone (DMQ₉), which is an intermediate in the synthesis of UQ₉ (Miyadera, H. et al., (2001). J. Biol Chem 276, 7713-6). Recent evidence suggests that clk-1 encodes a DMQ hydroxylase (Stenmark, P. et al., (2001). J. Biol Chem 2, 2). In E. coli, DMQ₈ is able to sustain respiration in isolated membranes although at a lower rate than UQ₈. Similarly, DMQ₉ is capable to convey electron transport in eukaryotic mitochondria, as the function of purified mitochondria (Felkai, S. et al., (1999). Embo J 18, 1783-92) and of mitochondrial enzymes (Miyadera, H. et al., (2001). J Biol Chem 276, 7713-6) from clk-1 mutants appear to be almost intact compared to the wild type. Furthermore, synthetic DMQ₂ can function as a co-factor for electron transport from complex I and, more poorly, from complex II (Miyadera, H. et al., (2001). J Biol Chem 276, 7713-6). Interestingly, only DMQ₉ is present in all three clk-1 alleles irrespective of the severity of their effect on physiological rates, which suggests that the lack of UQ cannot solely account for the Clk-1 phenotype (Miyadera, H. et al., (2001). J Biol Chem 276, 7713-6).

Recently, it has been found that clk-1 mutants are unable to grow on a UQ-deficient bacterial strain in spite of the presence and the activity of DMQ₉ (Jonassen, T. et al., (2001). Proc Natl Acad Sci USA 98, 421-6).

Although, dietary UQ is generally not capable to reach mitochondria, this has been interpreted to suggest that DMQ₉ is insufficient for normal mitochondrial function, and that dietary bacterial UQ₈ can reach the mitochondria and function there in trace amounts (Jonassen, T. et al., (2001). Proc Natl Acad Sci USA 98, 421-6).

It would be highly desirable to be provided with characterization of phenotypic effects of UQ deficiencies and screening methods for compounds that can affect the activity of clk-1 and/or relieve UQ deficiencies in multicellular organisms.

### SUMMARY OF THE INVENTION

In accordance with the present invention there is provided a method of screening for a compound allowing survival of clk1 homozygous mutant vertebrate embryos, which comprises the step of breeding heterozygous clk1 subjects to obtain clk1 homozygous mutant embryos and determining viability of clk1 homozygous embryos; wherein at least one of the heterozygous subject is treated with the compound prior to the breeding; and wherein viable embryos are indicative of a compound allowing survival of clk1 homozygous embryos.

The method in accordance with a preferred embodiment of the present invention, wherein the subject is a mouse.

The method in accordance with a preferred embodiment of the present invention, wherein the compound is suitable for partial or complete functional replacement of endogenous ubiquinone.

The method in accordance with a preferred embodiment of the present invention, wherein the compound is administered by at least one route selected from the group consisting of oral, intra-muscular, intravenous, intraperitoneal, subcutaneous, topical, intradermal, and transdermal route.

In accordance with the present invention, there is provided a method of screening for a compound suitable for rescue of mutant phenotype of mclk1 homozygous cell line, which comprises the step of determining a mutant phenotype in a mclk1 knockout cell line, wherein cell line is treated with the compound prior to the determining, and wherein the level of the phenotype is indicative of a compound suitable for rescue.

In accordance with the present invention, there is provided a method of screening for a compound suitable for partial or complete functional replacement of endogenous ubiquinone, which comprises the step of determining a mutant phenotype in a mclk1 knock-out homozygous ES cell line; wherein the cell line is treated with the compound prior to the determining; and wherein level of the phenotype is indicative a compound suitable for partial or complete functional replacement of ubiquinone.

The method in accordance with a preferred embodiment of the present invention, wherein the phenotype is cellular respiration and/or growth rate.

In accordance with the present invention, there is provided a method of screening for a compound suitable for partial or complete functional replacement of ubiquinone in a subject, which comprises the step of assessing at least one phenotype selected from the group consisting of viability, fertility, and total or partial absence of a mutant phenotype of a coq-3 homozygous mutant worm; wherein the worm is treated with the compound prior to the assessing; and wherein at least one phenotype selected from the group consisting of the viability, fertility and total or partial absence of the mutant phenotype is indicative of a compound suitable for partial or complete functional replacement of ubiquinone in the subject.

The method in accordance with a preferred embodiment of the present invention, wherein the compound is capable of reaching mitochondria in the subject.

In accordance with the present invention, there is provided a method for screening for a compound suitable for partial or complete functional replacement of ubiquinone in a subject, which comprises the step of assessing at least one phenotype selected from the group consisting of viability, fertility and total or partial absence of a Clk-1 phenotype of a clk-1 homozygous mutant worm grown on ubiquinone-depleted substrate; wherein the worm is treated with the compound prior to the assessing; and wherein at least one phenotype selected from the group consisting of the viability, fertility and total or partial absence of said Clk-1 phenotype is indicative of a compound suitable for partial or complete functional replacement of ubiquinone in the subject.

The method in accordance with a preferred embodiment of the present invention, wherein the ubiquinone-depleted substrate is a non-ubiquinone producer bacteria.

The method in accordance with another embodiment of the present invention, wherein the ubiquinone-depleted substrate is a bacteria producing ubiquinone having side-chains shorter than 8 isoprene units.

The method in accordance with another embodiment of the present invention, wherein the compound is capable of reaching at least non-mitochondrial sites of ubiquinone requirement in the subject.

The method in accordance with a preferred embodiment of the present invention, wherein the bacteria is selected from the group consisting of RKP1452, AN66, IS-16, DM123, GD1, DC349, JC349, JC7623, JF496, KO229(pSN18), KO229Y37A/Y38A), KO229(R321V), and KO229(Y37A/R321V).

The method in accordance with a preferred embodiment of the present invention, wherein the bacteria has a mutation in at least one of genes selected from the group consisting of ubiCA, ubiD, ubiX, ubiB, ubiG, ubiH, ubiE, ubiF, and ispB.

The method in accordance with a preferred embodiment of the present invention, wherein the bacteria carries at least one of the plasmids selected from the group consisting of pSNI8, Y37A/Y38A, R321V, Y37A/R321V.

The method in accordance with a preferred embodiment of the present invention, wherein the functional replacement of ubiquinone is for a function of ubiquinone as co-factor of CLK-1.

In accordance with the present invention, there is provided a method for screening a compound capable of inhibiting activity of clk-1 and/or other processes required to make ubiquinone from demethoxyubiquinone in a subject, which comprises the step of determining at least one phenotype selected from the group consisting of growth, fertility and total or partial absence of a Clk-1 phenotypes of a wild-type worm on a ubiquinone-depleted substrate; wherein the worm is treated with the compound prior to the determining; and wherein at least one phenotype selected from the group consisting of total or partial absence of growth, absence of fertility and total or partial absence of said Clk-1 phenotypes is indicative of a compound capable of inhibiting activity of clk-1 and/or other processes required to make ubiquinone from demethoxyubiquinone in a subject.

In accordance with the present invention, there is provided a method of screening for a compound suitable for complete or partial functional ubiquinone replacement, which comprises the step of determining a mutant phenotype of a subject in which mclk1 and/or a known ubiquinone biosynthetic enzyme gene is deleted and/or any other gene which when altered leads to absence or reduction of ubiquinone; wherein the subject is treated with the compound prior to the determining; and wherein level of the phenotype is indicative of a compound suitable for complete or partial functional ubiquinone replacement.

The method in accordance with a preferred embodiment of the present invention, wherein the subject is a mouse, ES cell line, or any cell line in which mclk1 is deleted or any gene coding for a known ubiquinone biosynthetic enzyme gene is deleted and/or any other gene which when altered leads to absence or reduction of ubiquinone.

In accordance with the present invention, there is provided a mouse which is incapable of producing ubiquinone and comprising a gene knock-out of mclk1; wherein the mouse expresses the phenotype related to an absence of ubiquinone and the presence of demethoxyubiquinone.

In accordance with the present invention, there is provided a DNA construct, which comprises an alteration of mclk1; wherein the DNA construct is instrumental in producing a mouse mclk1 knockout strain of the present invention.

In accordance with the present invention, there is provided an ES cell line which is incapable of producing ubiquinone and comprising a gene knock-out of mclk1; wherein the ES cell line expresses the phenotype related to an absence of ubiquinone and the presence of demethoxyubiquinone.

In accordance with the present invention, there is provided a coq-3 mutant subject which is incapable of producing ubiquinone; wherein mutation is a deletion of coq-3 or a deletion of a ubiquinone biosynthetic enzyme and/or any other gene which when altered leads to absence or reduction of ubiquinone.

The mutant in accordance with a preferred embodiment of the present invention, wherein the subject is a worm.

The mutant in accordance with a preferred embodiment of the present invention, wherein the mutant is selected from the group of worm identified using PCR primers selected from the group consisting of SHP172, SHP1773, SHP1774, SHP1775, SHP1840 and SHP1865.

In accordance with the present invention, there is provided a method of screening for a compound suitable for complete or partial functional ubiquinone or demethoxyubiquinone replacement, which comprises the step of determining a mutant phenotype in a subject in which a ubiquinone biosynthetic enzyme gene and/or any gene whose alteration leads to an absence or reduction of ubiquinone or demethoxyubiquinone is altered; wherein the subject is treated with the compound prior to the determining; and wherein level of phenotype is indicative of a compound suitable for complete or partial functional ubiquinone or demethoxyubiquinone replacement.

In accordance with the present invention, there is provided a method for reducing and/or increasing ubiquinone level in a multicellular subject, which comprises the step of targeting coq-3 in the subject.

In accordance with the present invention, there is provided a method of screening for a genetic suppressor of clk-1, which comprises the step of determining at least one phenotype selected from the group consisting of viability, fertility and total or partial absence of a Clk-1 mutant phenotype of clk-1 mutant worms grown on ubiquinone-depleted bacteria; wherein the worm carries the genetic suppressor prior to the determining; and wherein at least one phenotype selected from the group consisting of the viability, fertility and total or partial absence of said Clk-1 mutant phenotype is indicative of a genetic suppressor of clk-1.

In accordance with the present invention, there is provided a method of screening for a genetic suppressor of coq-3, which comprises the step of determining at least one phenotype selected from the group consisting of viability, fertility and total or partial absence of a mutant phenotype of coq-3 mutant worm; wherein the worm carries the genetic suppressor prior to the determining; and wherein the at least one phenotype selected from the group consisting of viability, fertility and total or partial absence of said mutant phenotype is indicative of a genetic suppressor of coq-3.

In accordance with the present invention, there is provided a method of screening for a compound suitable for complete or partial functional ubiquinone replacement, which comprises the step of determining a mutant phenotype of a subject in which mclk1 is deleted only in a subset of cells and/or periods of the life cycle, wherein the subject is treated with the compound prior to the determining; and wherein level of the phenotype is indicative of a compound suitable for complete or partial functional ubiquinone replacement.

The method in accordance with a preferred embodiment of the present invention, wherein the compounds are useful in treating a disease selected from the group consisting of reactive oxygen species (ROS) mediated disease, diabetes, hypoxia/reoxygenation injury, Parkinson's disease, artherosclerosis and Alzheimer's disease.

In the present application, the term “ubiquinone-depleted substrate” is intended to mean a substrate being not producing ubiquinone or being producing ubiquinone with side-chains too short to be effective. An example of what will be considered ubiquinone with side-chains too short to be effective would be ubiquinone with side-chains shorter than 8 isoprene units.

### BRIEF DESCRIPTION OF THE DRAWINGS

FIG. 1 illustrates the coq-3 gene and its deletion in coq-3(qm188);

FIGS. 2A-E illustrate the targeted disruption of the mouse mclk1 gene;

FIG. 3 illustrates the severe developmental delay in mclk1 mutant embryos;

FIGS. 4A-C illustrate the generation of the mclk1^{flox} allele. Analysis by Southern blot on neomycin resistant clones;

FIG. 5 illustrates the comparison of COQ-3 proteins from different species (SEQ ID NOS: 3-6);

FIGS. 6A-E illustrate the Mus musculus genomic sequence of mclk-1 (Exons are in bold) (SEQ ID NO: 15);

FIGS. 7A-E illustrate the Mus musculus genomic sequence in mutant knock-out allele of mclk-1 (Exons are in bold, neomycin cassette is in lowercase) (SEQ ID NO: 16); and

FIGS. 8A-E illustrates the sequence of mclk1^{flox} allele. (Exons in bold, loxp sequence in italic, DNA fragment inserted underlined.) (SEQ ID NO: 21)

### DETAILED DESCRIPTION OF THE INVENTION

In accordance with the present invention, there is provided characterization of phenotypic effects of ubiquinone deficiencies in multicellular organisms.

Ubiquinone is Necessary for C. elegans Development and Fertility

clk-1 mutants are incapable of completing development when fed on an ubiG E. coli mutant strain (Jonassen, T. et al., (2001). Proc Nati Acad Sci U S A 98, 421-6), which produces no ubiquinone (UQ). The ubiG gene product is required at two steps of the UQ biosynthesis pathway, and ubiG mutants do not produce any UQ. Tests were performed to verify whether this growth phenotype resulted from a specific toxicity of the ubiG strain (GD1) for clk-1 mutants, or from the absence of UQ. For this purpose, a systematic analysis of the growth of clk-1 mutant worms on a variety of E. coli mutants that are defective for UQ biosynthesis (ubi mutants) was conducted. Nine E. coli enzymes have been described as participating in UQ biosynthesis. They are all membrane-bound, except the first one, ubiC, which is a soluble chorismate lyase. The next enzyme in the pathway is the prenyltransferase ubiA that attaches the isoprenoid side chain to the quinone ring (8 subunits in E. coli). The other enzymes are grouped in three categories: decarboxylases (ubiD, ubiX), monooxygenases (ubiB, ubiH, ubiF), and methyltransferases (ubiG, ubiE). Standard procedures were used for bacterial and worm cultures, except that the NGM plates contained 0.5% glucose, to minimize the reversion of UQ-deficient strains. To evaluate the development of worms on various bacterial strains, adult hermaphrodites were picked and bleached on a plate containing the test bacteria, following standard methods. This step ensures that no OP50 bacteria contamination is present on the test plate. L1 larvae that hatched from the bleached eggs were transferred to a fresh plate, and the growth of the worms was examined. The genotypes of the bacterial strains used are described in Table 1. The growth of the three clk-1 mutant strains on strains of bacteria mutant for each of these genes was examined (Table 1). Three clk-1 mutant alleles have been identified: qm30 and qm51, which are putative nulls, and e2519, which carries a point mutation in the clk-1 gene and displays a relatively milder phenotype.

TABLE 1
E. coli strains used

| Strain | Genotype |
|---|---|
| OP50 | ura |
| RKP1452 | Km^{R}, ΔubiCA::Km^{R} |
| AN66 | thr-1 leuB6 ubiD410 |
| IS-16 | ubiX, derived from the THU strain |
| DM123 | RM1734 yigR::Kan |
| GD1 | ubiG::Kan |
| DC349 | FadR mel adhC81 acdA1 |
| AN70 | Hfr metB StrR ubiE-401 |
| JC7623Δ4-1 | JC7623, ubiE::KanR |
| JF496 | ubiF411 asnB50::Tn5 |

It was found that on all the bacterial ubi−(mutant) strains tested, L1 larvae from the wild-type strain N2 are capable of completing development to adulthood and these adults have a brood-size of approximately 320, which is similar to their brood size on .ubi+bacteria (OP50) (Table 2). This indicates that endogenously synthesized UQ is sufficient to maintain a wild-type phenotype, without a requirement for dietary UQ. A number of worm mutants that are not known to be involved in UQ synthesis (dpy-9, eat-2, mau-2), including long-lived mutants (daf-2 and a number of strains that show a Clk-1-like phenotype that have not been fully characterized) were examined. In no case was the growth of the mutants impaired on ubi−bacteria. In contrast, all three clk-1 mutants behave identically on most ubi−bacterial strains tested: they develop very slowly, or not at all, and produce no progeny (Table 2). However, the clk-1 mutants can develop and produce some progeny on ubiD, ubiX and ubiH mutant strains, which are point mutants producing residual amounts of ubiquinone (around 15% of the wild type). Thus, the relatively low levels of bacterial UQ₈ are sufficient to allow for the growth of clk-1 mutants.

TABLE 2
Growth and brood-size analysis of wild-type and clk worms
on ubi + and ubi − bacteria

| | | N2 (Wild type) | | clk-1 mutants | |
|---|---|---|---|---|---|
| Strain | Genotype | Growth | Progeny | Growth | Progeny |
| OP50 | ubi + | + | 323 ± 16 | + | qm30: 94 ± 12 |
| | | | | | qm51: 83 ± 10 |
| | | | | | e2519: 177 ± 4 |
| RKP1452 | ubiCA KO | + | 331 ± 37 | − | 0 |
| AN66 | ubiD | + | 313 ± 16 | + | qm30: 82 ± 5 |
| | | | | | qm51: 93 ± 6 |
| | | | | | e2519: 182 ± 26 |
| IS-16 | ubiX | + | 336 ± 8 | + | qm30: 96 ± 11 |
| | | | | | qm51: 83 ± 10 |
| | | | | | e2519: 164 ± 8 |
| DM123 | ubiB KO | + | 312 ± 25 | − | 0 |
| GD1 | ubiG KO | + | 315 ± 15 | − | 0 |
| DC349 | ubiH | + | 329 ± 16 | + | qm30: 105 ± 6 |
| | | | | | qm51: 90 ± 3 |
| | | | | | e2519: 168 ± 11 |
| JC7623 | ubiE KO | + | 313 ± 4 | − | 0 |
| JF496 | ubiF | + | 330 ± 4 | − | 0 |

C. elegans is Sensitive to Ubiquinone Side-Chain Length Ubiquinone (UQ) is composed of a quinone ring and an isoprenoid chain, whose length is species-specific. There are 9 isoprene repeats in C. elegans, 8 in E. coli, and 6 in S. cerevisiae. In mammals, both UQ₉ and UQ₁₀ are detected (the subscript refers to the length of the isoprenoid side chain). UQ₁₀ is the major UQ species present in humans, while UQ₉ is predominant in mice and rats (Dallner, G. and Sindelar, P. J. (2000). Free Radic Biol Med 29, 285-94). The differential tissue distribution of UQ₉ and UQ₁₀ is presented in Table 3 (Dallner, G. and Sindelar, P. J. (2000). Free Radic Biol Med 29, 285-94).

TABLE 3
Ubiquinone tissue distribution in rat and human

| | Rat | | | Human | | |
|---|---|---|---|---|---|---|
| | UQ₉ | UQ₁₀ | | UQ₉ | UQ₁₀ | |
| | μg/g | μg/g | UQ₁₀/UQ₉ | μg/g | μg/g | UQ₉/UQ₁₀ |
| | tissue | tissue | (%) | tissue | tissue | (%) |
| Heart | 202 | 17 | 8 | 3 | 114 | 2.5 |
| Liver | 131 | 21 | 14 | 2 | 55 | 3.5 |
| Kidney | n.d | n.d | — | 3 | 67 | 4.5 |
| Brain | 37 | 19 | 34 | 1 | 13 | 7 |
| Spleen | 23 | 9 | 28 | 1 | 25 | 4 |
| Lung | 17 | 2 | 10.5 | 1 | 8 | 11 |
| Intestine | 51 | 19 | 27 | n.d | n.d | — |

The length of the UQ side-chain is controlled by polyprenyl-diphosphate synthases. These enzymes are encoded by essential genes, and have been cloned in many organisms, including S. cerevisiae (coq1: hexaprenyl-diphosphate synthase), E. coli (ispB: octaprenyl-diphosphate synthase), and Rhodobacter capsulatus (sdsA: solanesyl-diphosphate synthase).

To evaluate the importance of UQ side-chain length, using C. elegans. The exogenous UQ fed to the worms was manipulated by exposing the worms to an E. coli mutant strain where the original ispB gene is knocked-out, and replaced by different versions of ispB carried on rescuing plasmids. The ispB version dictates the side-chain length of the bacterially-manufactured UQ. N2 (Bristol) was used as wild-type strain, and analyzed clk-1 (qm30), clk-1 (qm51), clk-1 (e2519) and daf-2(e1370) mutants strains. The genotypes of the bacterial strains used are described in Table 4. The plasmids encoding mutant versions of ispB are described in Table 5. Table 6 is providing the results obtained from brood size measurements. The entire progeny of 10 worms was counted and the experiment was performed twice.

TABLE 4
Genotypes of the bacterial strains used in the
study of the effect of UQ side-chain length

| Strain | Genotype | Reference |
|---|---|---|
| OP50 | ura | Laboratory collection |
| KO229 | ispB::Camr | Okada et al., 1997* |

*Okada et al., (1997). Journal of bacteriology, 179, 9, 3058-3060

TABLE 5
Plasmids encoding versions of ispB

| | | Major UQ | Minor UQ | |
|---|---|---|---|---|
| Plasmid | Characteristics | produced | produced | Reference |
| pSN18 | Amp^{r}, encodes | UQ₉ | — | Okada et |
| | Rhodobacter | | | al., 1997* |
| | capsulatus ispB | | | |
| | homolog (sdsA) | | | |
| Y37A/ | Amp^{r}, encodes a | UQ₇ | UQ₈, UQ₆ | Kainou et |
| Y38A | mutant version of E. | | | al., 2001** |
| | coli ispB gene | | | |
| R321V | Amp^{r}, encodes a | UQ₆ | UQ₇, UQ₅ | Kainou et |
| | mutant version of E. | | | al., 2001 |
| | coli ispB gene | | | |
| Y37A/ | Amp^{r}, encodes a | UQ₆ | UQ₇, UQ₅ | Kainou et |
| R321V | mutant version of E. | | | al., 2001 |
| | coli ispB gene | | | |

*Okada et al., (1997). Journal of bacteriology, 179, 9, 3058-3060
**Kainou et al., (2001). The Journal of Biological Chemistry 276, 11, 7876-7883

TABLE 6
Brood-size analysis

| | | clk-1 | clk-1 | clk-1 | daf-2 |
|---|---|---|---|---|---|
| | N2 | (qm30) | (qm51) | (e2519) | (e1370) |
| OP50 (UQ₈) | 240, 282 | 94, 108 | 112, 121 | 158, 181 | 254, 228 |
| pSN18 | 266, 246 | 107, 102 | 94, 117 | 170, 193 | 249, 264 |
| (UQ₉) | | | | | |
| Y37A/Y38A | 255, 291 | 0, 0 | 0, 0 | 149, 178 | 266, 237 |
| (UQ₇) | | | | | |
| R321V | 236, 258 | 0, 0 | 0, 0 | 82, 93 | 218, 259 |
| (UQ₆) | | | | | |
| Y37A/ | 247, 273 | 0, 0 | 0, 0 | 95, 101 | 241, 229 |
| R321V | | | | | |
| (UQ₆) | | | | | |

Growth Rate on Various Bacterial Strains

Post-embryonic growth of the worms on the various bacterial strains was qualitatively evaluated. It was observed that N2 and daf-2 mutants grow at similar rates on all bacterial strains. However, clk-1 (qm30) mutants had a similar growth rate on OP50 and KO229(pSN18), but were delayed by 3-5 days on the other strains. Also, the post-embryonic development of clk-1(e2519) mutants was delayed by ˜1 day on KO229(R321) mutants, as compared to OP50. Their growth on KO229(Y37AIY38A) is less severely affected. Finally, the onset of egg laying by clk-1 (e2519) was delayed by 1 day on KO229(Y37A/Y38A) and by 3 days on KO229(R321A) and KO229(Y37A/R321 A).

Thus, these experiments revealed a process in C. elegans that is sensitive to ubiquinone side-chain length as indicated by the behaviour of clk-1 mutants on bacterial strains that produce short chain ubiquinones. An inappropriate chain length severely alters development and fertility in qm30 and mildly or not at all in e2519.

The observation that clk-1 (e2519) mutants are almost unaffected in spite of the fact that they are known to produce no detectable ubiquinone, indicates that CLK-1 participates in processes that are different from ubiquinone synthesis. One can also infer that the e2519 mutation does not greatly affect this additional function or functions of CLK-1. However, these processes are ubiquinone-dependent as clk-1 (e2519) mutants cannot develop in the total absence of ubiquinone. For example, ubiquinone could act as a redox co-factor in these processes.

Endogenous Ubiquinone is Necessary for C. elegans Development and Fertility

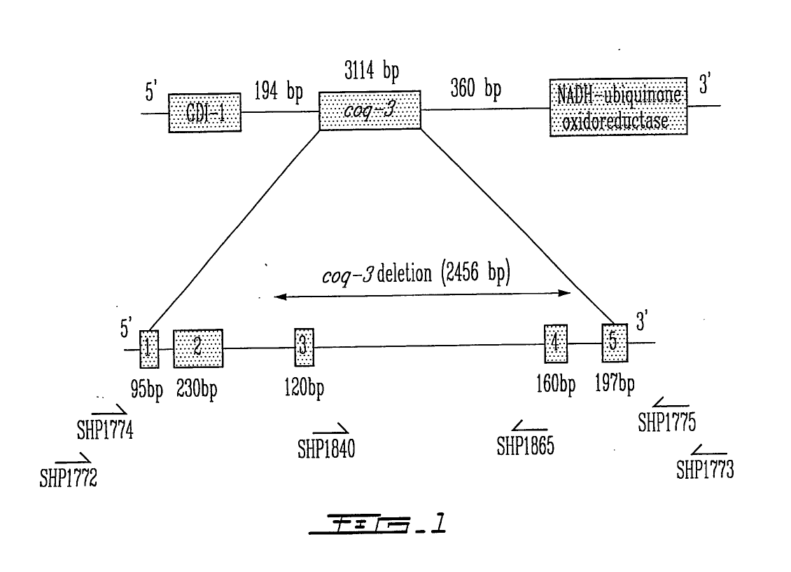

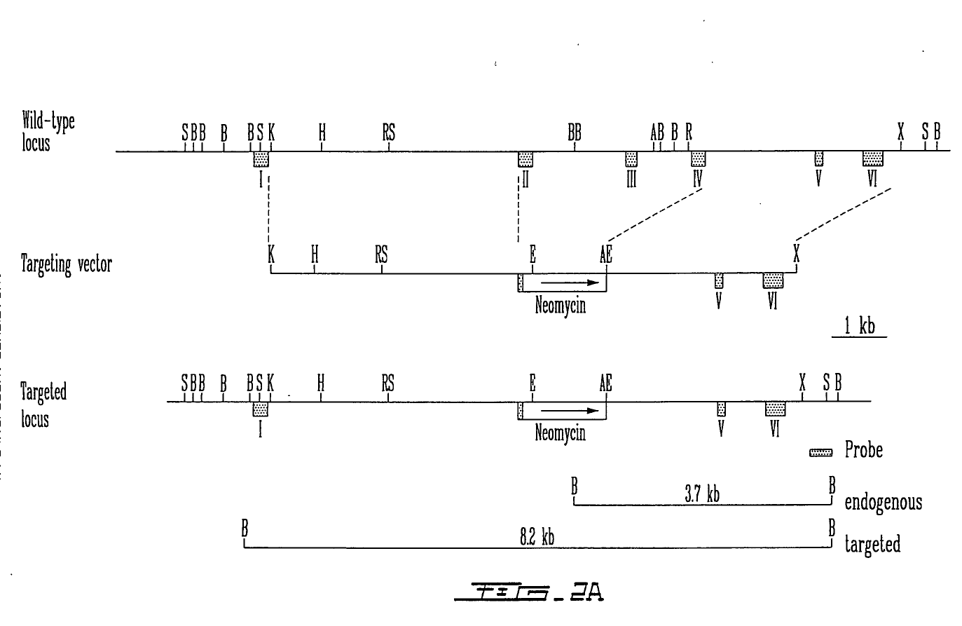

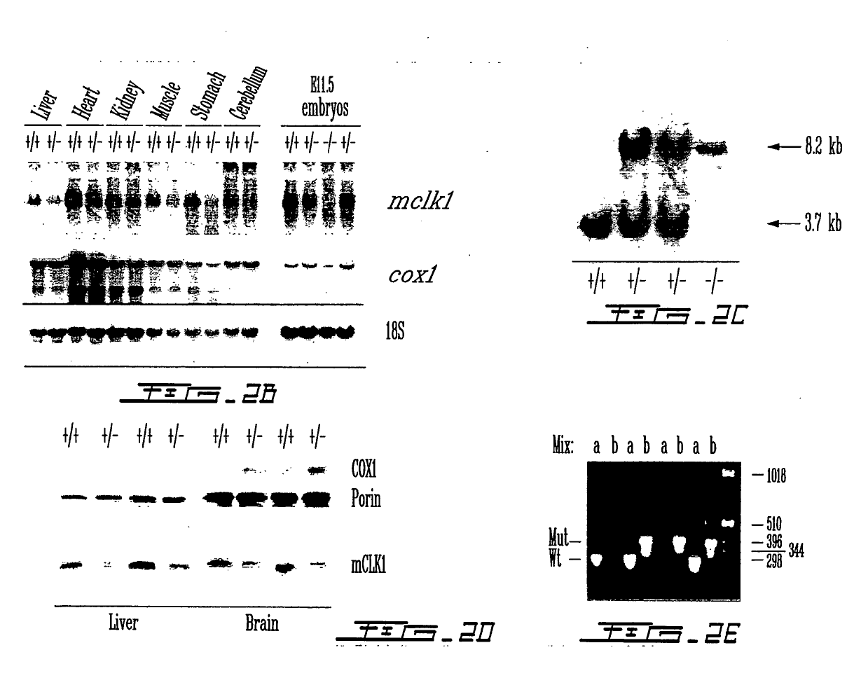

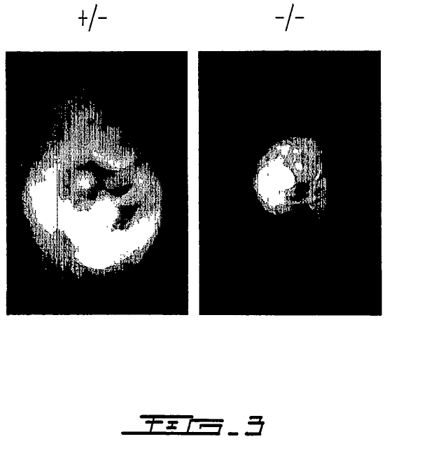

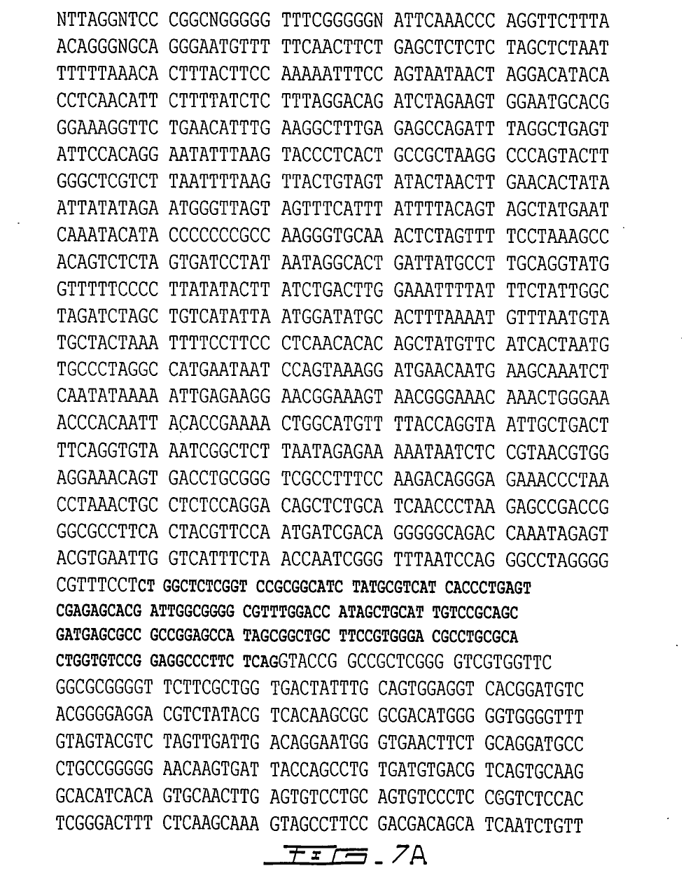

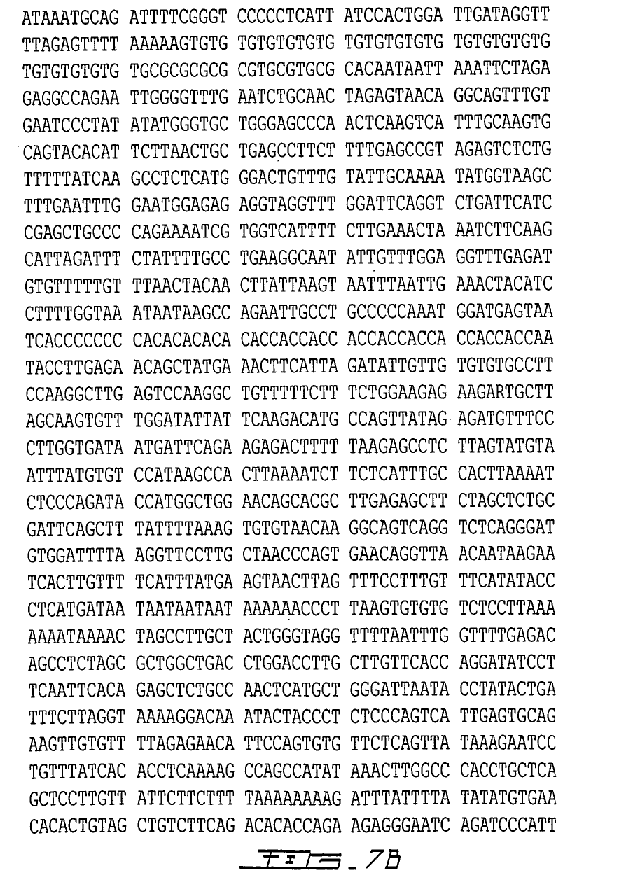

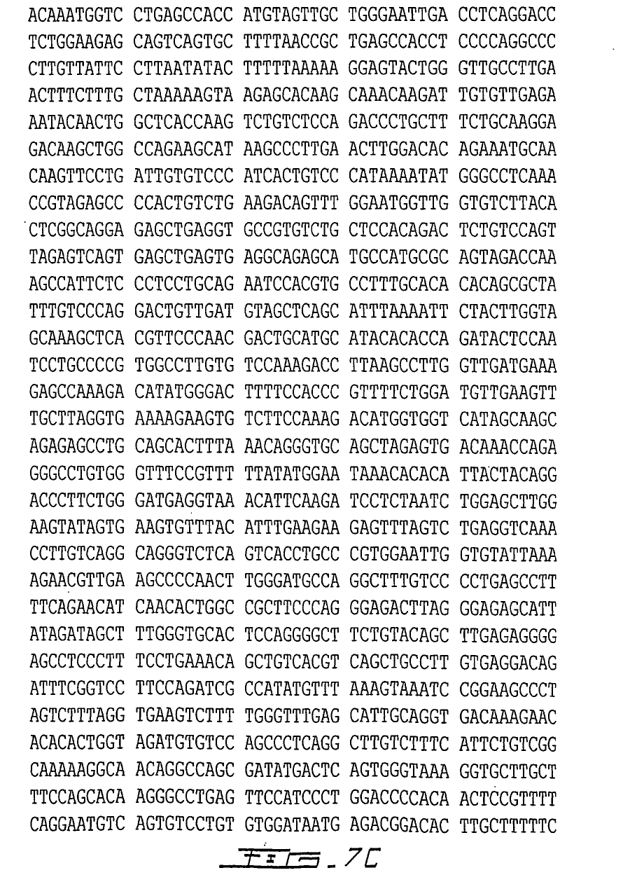

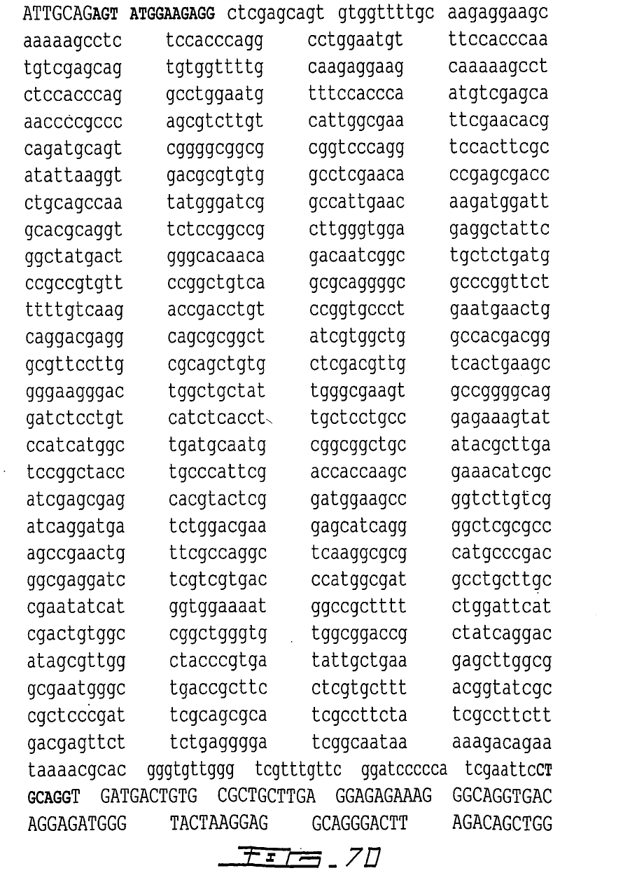

To test whether dietary UQ is sufficient for C. elegans development, a knockout mutation of the worm gene coq-3 was produced (SEQ ID NO:1). coq-3 encodes a methyltransferase (SEQ ID NO:2) whose homologues (Coq3p and UbiG) have been extensively characterized in the yeast S. cerevisiae and in E. coli, respectively. The enzyme acts at two different steps of Q synthesis and neither UQ nor DMQ is produced in the yeast and bacterial mutants. The worm COQ-3 protein is 29% identical to S. cerevisiae Coq3p and 28% to E. coli UbiG (FIG. 5 and SEQ ID NOS:3-6). A method of random mutagenesis and PCR-based screening was used to identify a deletion in coq-3 adapted from a standard protocol. The coq-3 gene is located on chromosome 4 of C. elegans, and as shown in FIG. 1, is part of an operon, comprising the gdi-1 gene and the NADH-ubiquinone oxidoreductase gene. coq-3 contains five predicted exons. The deletion in coq-3(qm188) removes 2456 bp (SEQ ID NO:7), and thus eliminates exons 3 and 4 (SEQ ID NOS: 1 and 8), and prevents any functional protein to be produced. To verify the genotype of coq-3, PCR analysis was performed, and used sets of primers whose priming regions are either outside of the coq-3 gene, or inside the region corresponding to the deletion obtained in the qm188 mutation. To check the presence of a deletion in the coq-3 gene, PCR analyses were carried out using genomic DNA from single worms. Each DNA preparation was simultaneously tested with primers recognizing sequences either outside the coq-3 gene (SHP 1772 (5′-CTGATTTCTTCCAGAGCTCTCTTGCCGCAC3′) (SEQ ID NO: 9), SHP 1773 (5′-AGCATTCCQGAGATGATGCACTCCTTGAGG-3′) (SEQ ID NO: 10), SHP 1774 (5′-TAGCGACTCTCAGCGACAAGCTTMCC-3′) (SEQ ID NO: 11) and SHP 1775 (5′-GAGGCCGGTTCCGAGACGATGGCATCG-3′) (SEQ ID NO: 12)), or inside the obtained deletion (SHP 1840 (5′-CCTCCTCGCGCACTACACACCATC-3′) (SEQ ID NO: 13) and SHP 1865 (5′-CGMGCGACGACTGCATCGTAGGC-3′) (SEQ ID NO: 14)). FIG. 1 displays the primers' localization. When using primers amplifying the whole coq-3 gene, a band of 4.3 kb was obtained with a wild-type worm. In contrast, a mutant band was amplified at 1.8 kb from a coq-3/coq-3 worm. When using primers annealing in the deletion region, both wild-type and heterozygote worms gave a PCR product of 1.1 kb, while no band was detected from a coq-3/coq-3 homozygote worm, which confirmed the homozygote nature of coq-3/coq-3 mutants.

Self-fertilizing coq-3(qm188)/+hermaphrodites produce ¼ of homozygous coq-3(qm188)/coq-3(qm188) progeny, as verified by PCR. These coq-3 homozygotes develop slowly and appear substantially smaller than wild-type worms. Most are sterile, but approximately 25% (n=31) produce some progeny (5-10 eggs) that arrests at the L1 stage and die quickly thereafter. For brood-size measurements, the entire progeny of 20 worms was counted. These observations indicate a partial maternal rescue effect of coq-3 homozygotes by the heterozygous mothers, as the phenotype of the first homozygous generation (slow development to adulthood) is less severe than that of the second homozygous generation (arrest at the L1 stage). UQ provided to the embryo by the mother or to maternal deposits of coq-3 mRNA or protein can provide the maternal effect.

It is also observed that the brood size of heterozygous coq-3/dpy-4 worms was much reduced (185±64; n=20) suggesting that the level of coq-3 expression might be limiting for UQ biosynthesis and that the worm's reproductive capacity is very sensitive to reduced level of endogenous UQ biosynthesis.

To ascertain that the observed phenotypes are solely due to the mutation in the coq-3 gene, the genomic fragment corresponding to the wild-type coq-3 gene was introduced into coq-3/+ heterozygotes using the rol-6 transformation marker by germline transformation. The micro-injection procedure was followed to generate standard extrachromosomal arrays. A PCR fragment (50 ng/μL) comprising the coq-3 genomic sequence was injected to assay for rescue. pRF4 plasmid (120 ng/μL) was used as a co-injection marker to screen for transgenic worms. coq3/dpy4 worms were utilized for injection since coq-3 homozygotes are lethal. The homozygous rescued lines were selected by checking the absence of the Dpy phenotype in their progeny, and the genotype was confirmed by PCR analysis.

Homozygous coq-3 transgenic animals (displaying the marker phenotype, Rol) develop normally and are fertile, indicating that the phenotype observed is indeed due to the coq-3 deletion. However, the extrachromosomal array carrying the coq-3 and rol-6 sequences is incapable of producing a strong maternal effect. Indeed, homozygous animals without the array (phenotypically non-Rol) issued directly from mothers carrying the array (phenotypically Rol) did not develop beyond the L2 stage. The expression of genes from extrachromosomal arrays is sometimes silenced and is poor in the C. elegans germline. The observation of a maternal effect indicates that the mother deposits an essential product in the oocytes (UQ and/or coq-3 mRNA). In either case, proper expression of coq-3 in the germline is necessary for the effect.

The lethal phenotype of coq-3 mutants indicates that dietary UQ is not sufficient for the growth and development of worms. This is consistent with findings in other systems that indicate that dietary UQ cannot reach the mitochondrial compartment, or only in extremely small amounts. The possibility that dietary UQ could be sufficient for worms was proposed to account for the viable phenotype of clk-1 mutants grown on ubi+bacteria, and their lethal phenotype when grown on ubi−mutant bacteria. However, the phenotype of coq-3 mutants clearly indicates that even in the presence of dietary bacterial UQ₈, a total absence of endogenous UQ₉ and DMQG (in coq-3 mutants) is not equivalent to the replacement of endogenous UQ₉ by endogenous DMQG (in clk-1 mutants).

In this context, it is of particular interest that clk-1 mutants cannot thrive by feeding on ubiF mutants. Indeed, UQ biosynthesis in ubiF mutants is blocked at the same level as in clk-1 mutants, and ubiF bacteria thus produce DMQ₈. As DMQ₉ performs efficiently in the mitochondrial respiratory chain (Miyadera et al., 2001), our findings demonstrate that neither endogenous nor dietary DMQ can replace UQ at non-mitochondrial sites of UQ requirement.

Ubiquinone is Necessary at Mitochondrial and Non-Mitochondrial Sites

The results presented here demonstrate that UQ is necessary for C. elegans growth and development at different subcellular locations. First, in the mitochondria, endogenous DMQ₉ can functionally replace endogenous UQ₉. Indeed, clk-1 mutant mitochondria do not contain UQ₉ but are functionally competent (Miyadera, H. et al., (2001). J Biol Chem 276, 7713-6), and the phenotype of coq-3 mutants, which produce neither UQ₉ nor DMQ₉, is much more severe than that of clk-1 mutants. Second, at non-mitochondrial sites, endogenous DMQG or dietary DMQ₈ or dietary UQ with a side-chain length shorter than 8 isoprene units cannot functionally replace endogenous UQ₉, while dietary UQ₈ can. In fact, clk-1 mutants, which have functional mitochondria and make DMQ₉, cannot develop and grow without dietary UQ₈, even in the presence of dietary DMQ₈ from ubiF bacteria or dietary UQ with a short side-chain.

This is consistent with the findings by numerous studies on UQ uptake and metabolism in other systems, such as rodents (Dallner, G. and Sindelar, P. J. (2000). Free Radic Biol Med 29, 285-94). Dietary UQ in these experiments appears to be taken up only poorly (2-3% of the initially ingested ubiquinone) and the majority is then distributed to the plasma membrane, the lysosomes and the golgi, with only minute quantities, if at all, appearing in the mitochondria. Given that every cell endogenously produces UQ, no active uptake system has been identified to assimilate this rather complex lipid.

These studies clarify the roles of endogenous and dietary UQ in the worm's biology. Also, for the first time it demonstrated the functional importance of UQ at non-mitochondrial locations for an organism's viability or fertility. Action of dietary UQ at non-mitochondrial sites could underly the beneficial effects of dietary UQ for patients with mitochondrial diseases (Dallner, G. and Sindelar, P. J. (2000). Free Radic Biol Med 29, 285-94). For example, UQ has been found to participate in reactions that regulate the redox state of the cell at the plasma membrane. Disease states which arise from defident mitochondria are often found to increase cellular oxidative stress and dietary UQ could stimulate a protective function at the plasma membrane. In addition, in bacteria, quinones have been found to act as the primary signal of the redox state of the cell. In E. coli, UQ negatively modulates the phosphorylation status and function of ArcB, an important global regulator of gene expression.

The coq-3 and clk-1 mutant strains provide genetic systems to identify compounds that selectively replace ubiquinone at the mitochondria and/or at non-mitochondrial sites. Screens for such compounds can be based on their ability to rescue selectively the phenotypes of coq-3 or clk-I mutants grown on UQ defident bacteria or not. For example, compounds that can reach the mitochondria, should rescue the phenotype of coq-3 mutants.

On the other hand, compounds selective for sites outside the mitochondria should rescue the phenotype of clk-1 worms grown on UQ-deficient bacteria, but should not rescue the lethal phenotype of coq-3 animals grown on wild-type bacteria. The development of such bio-available ubiquinone mimetics is of great medical interest.

Study of the Phenotypic Consequences of a Disruption in the Gene mclk-1 of Mus musculus

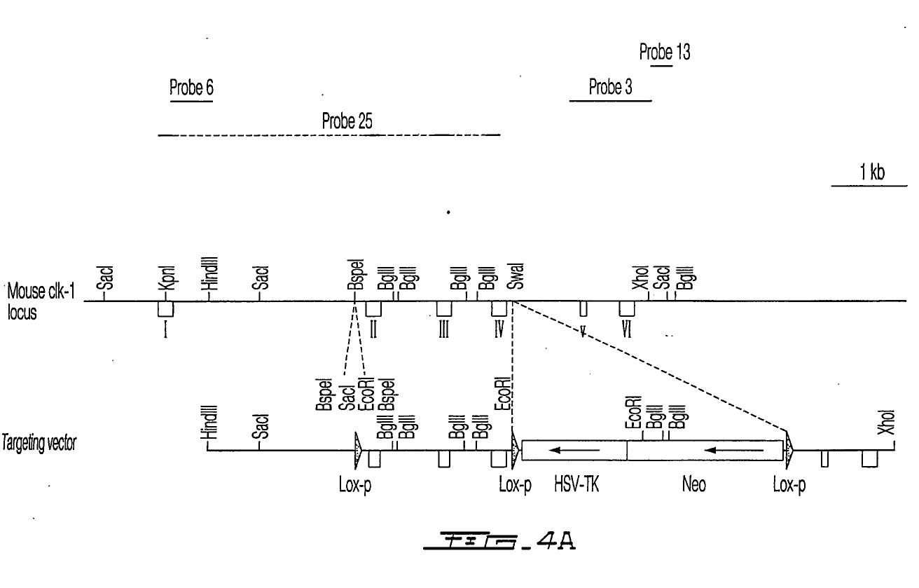

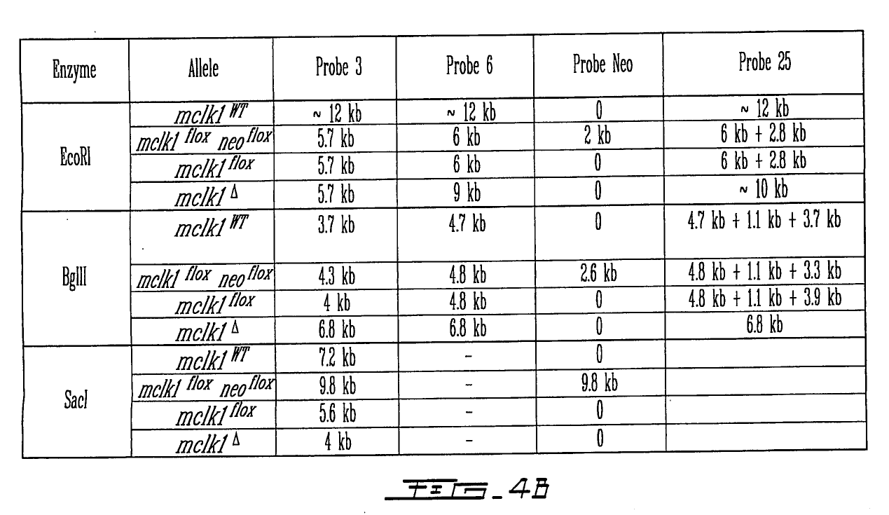

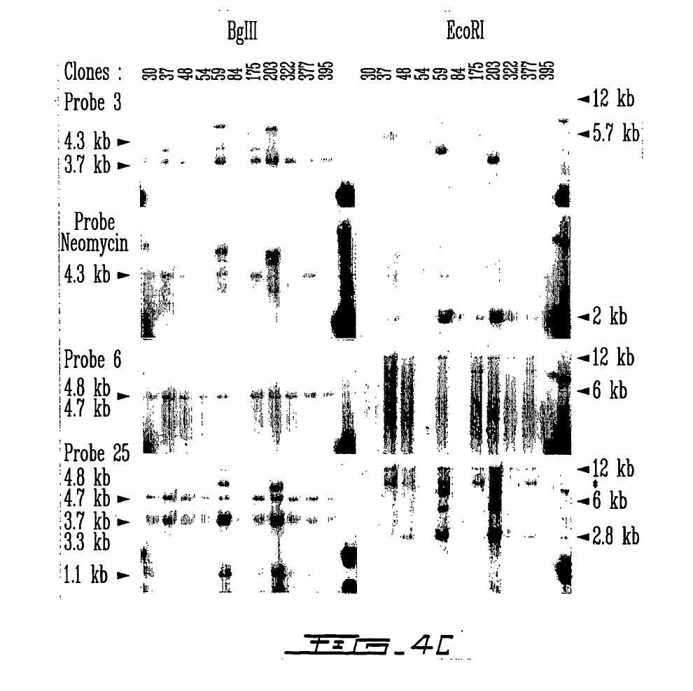

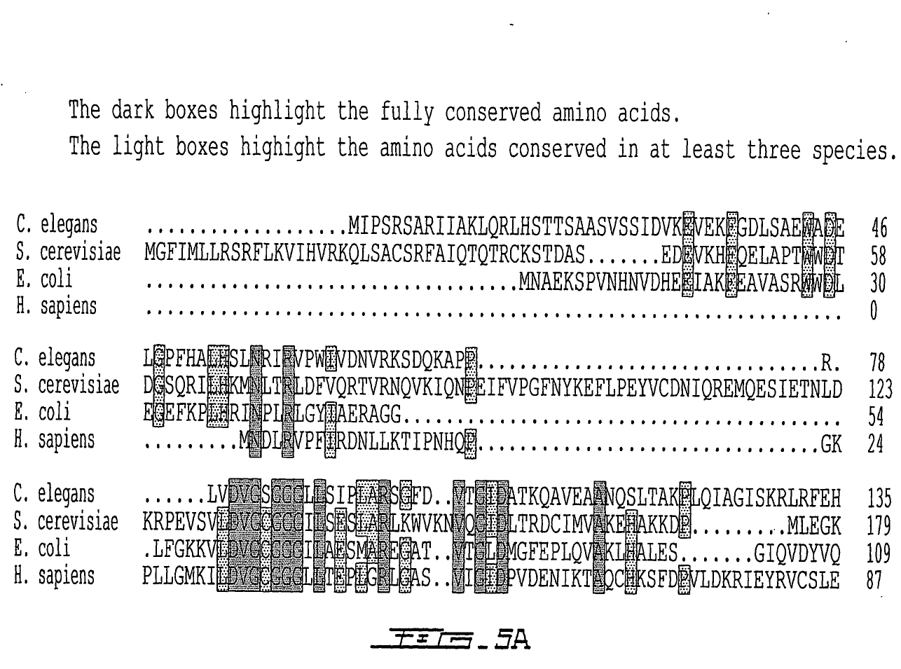

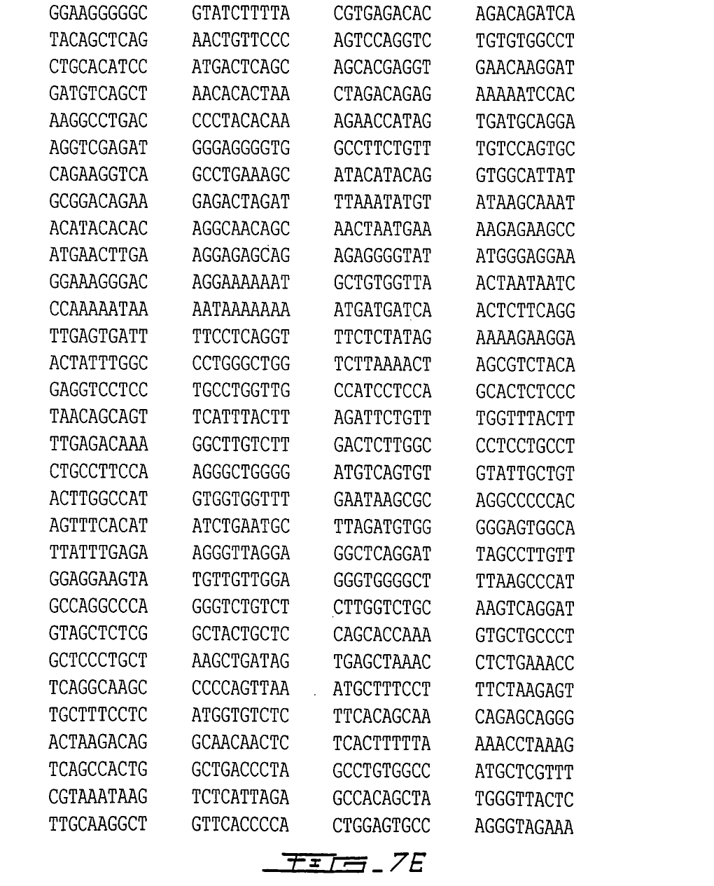

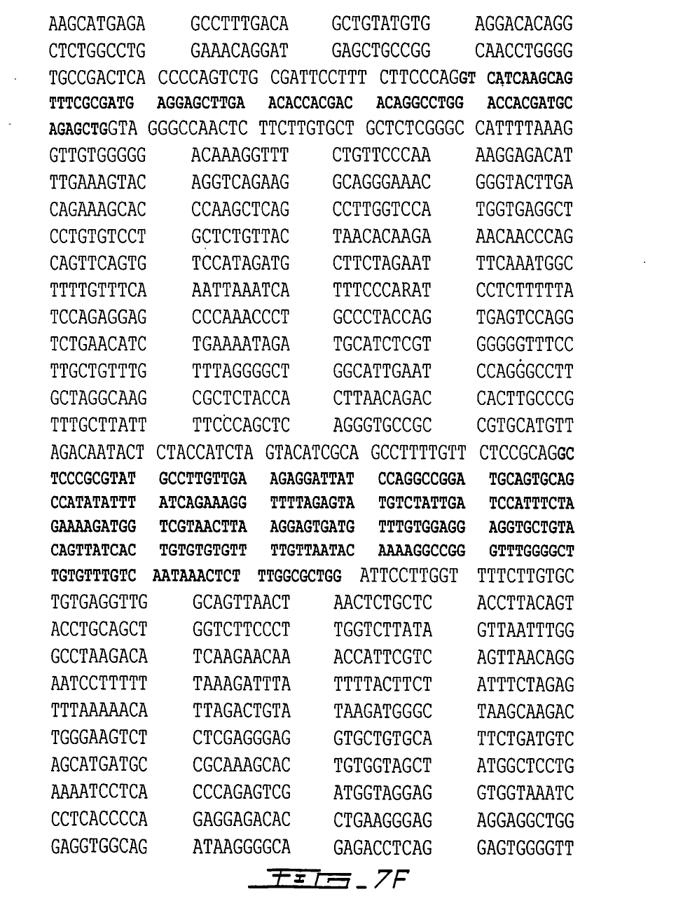

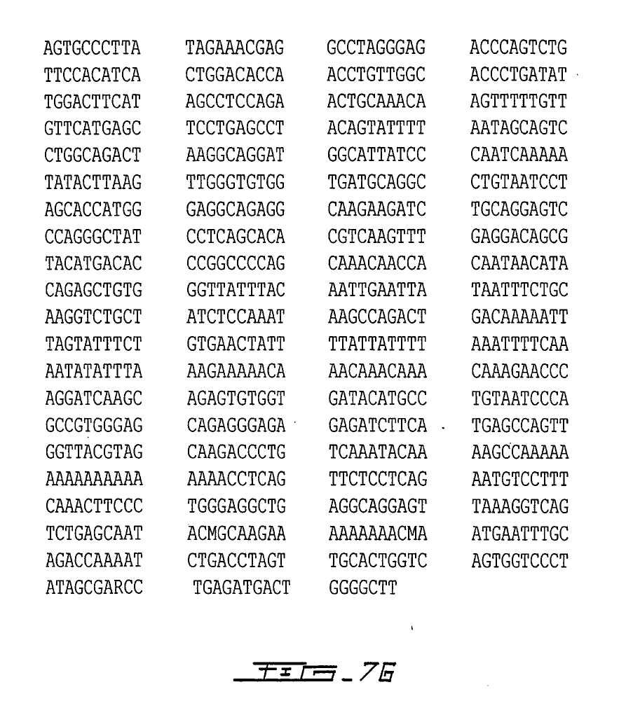

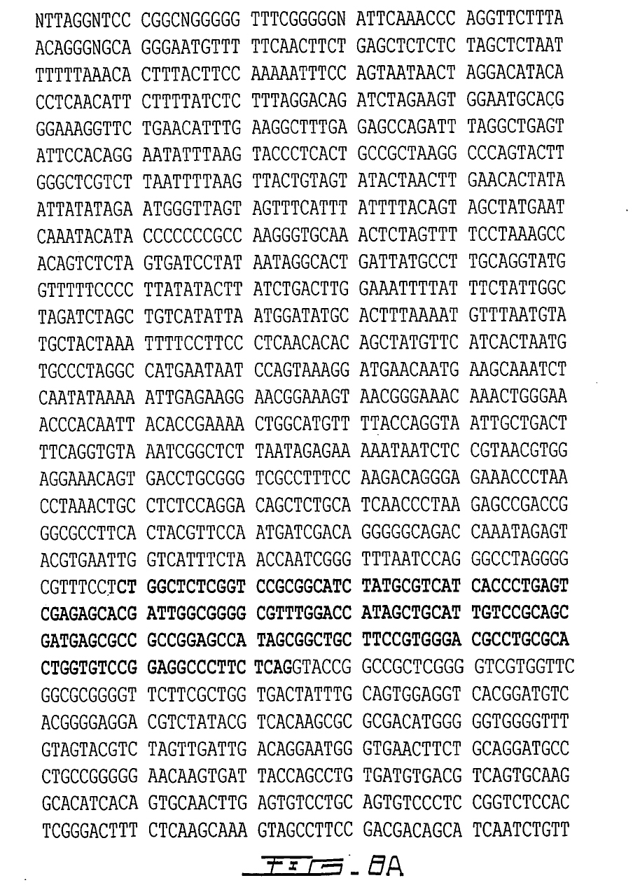

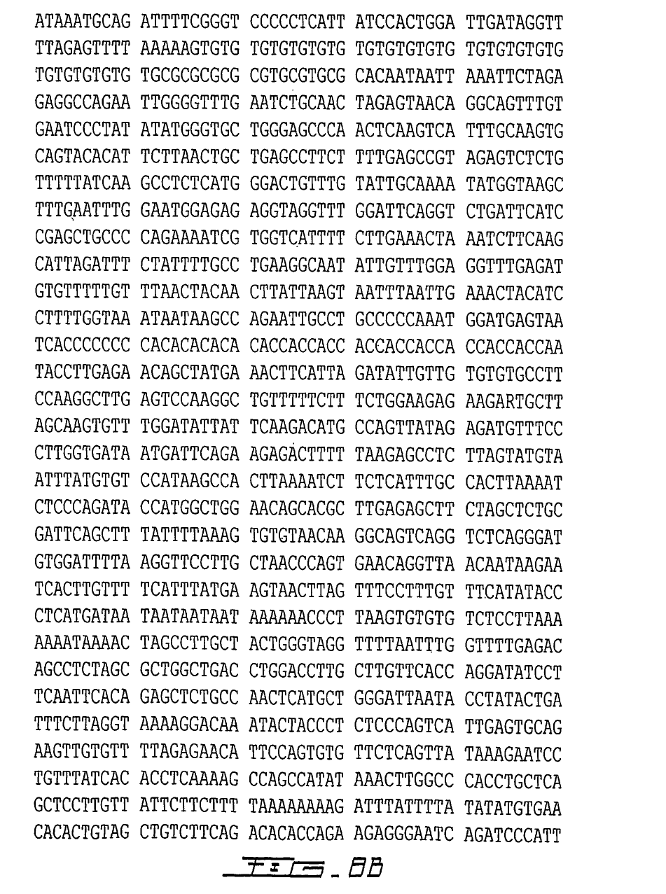

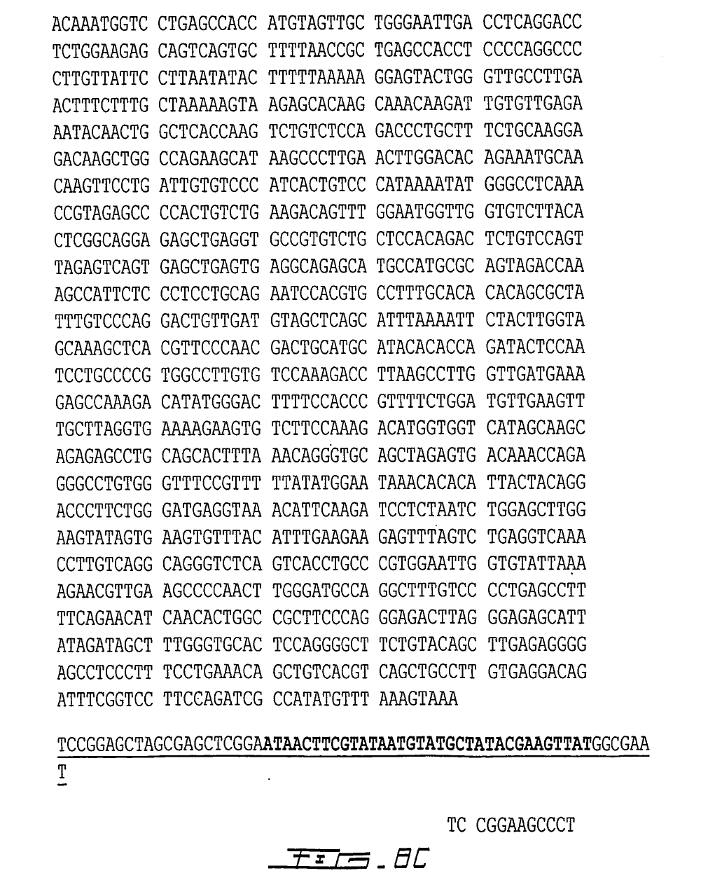

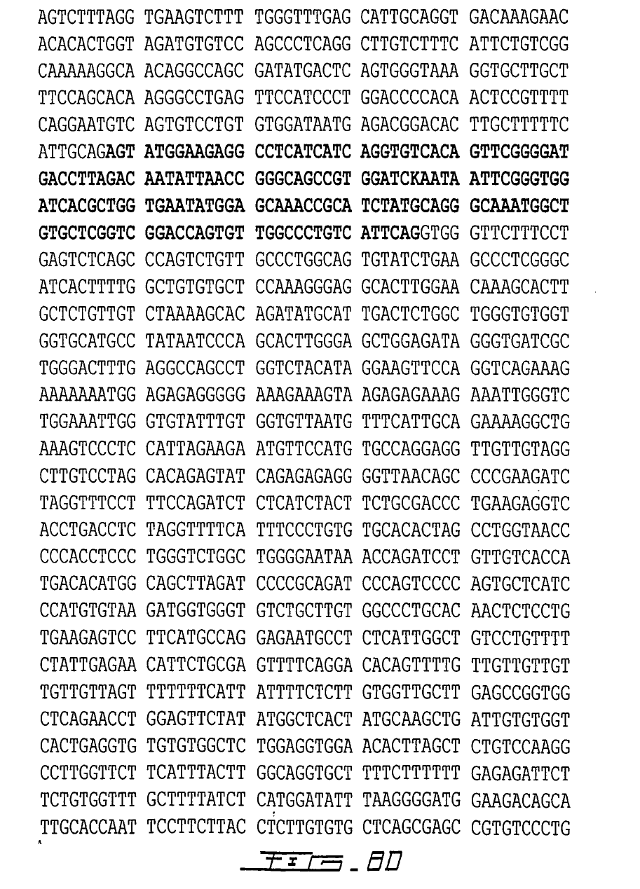

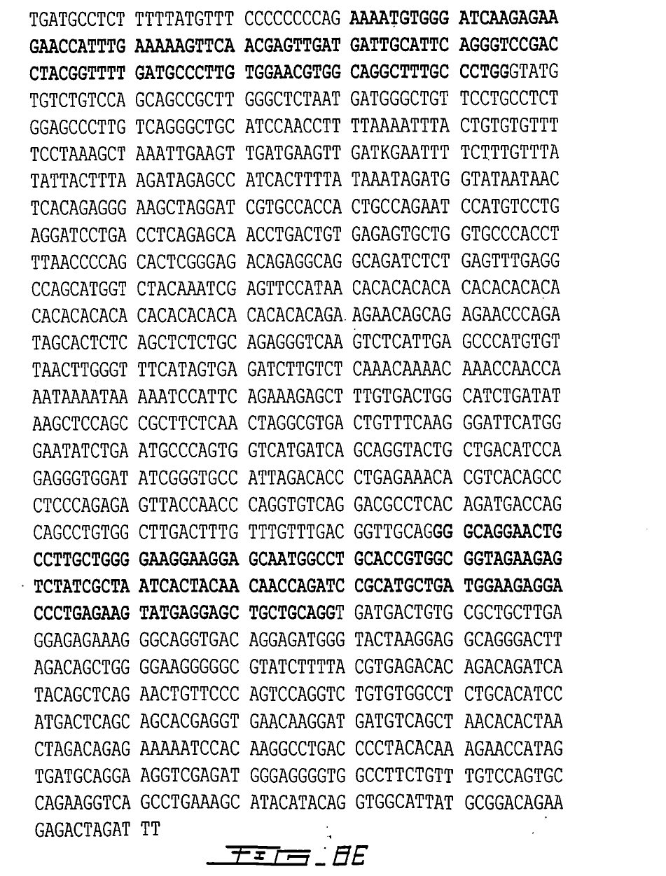

The mclk1 locus was disrupted in murine embryonic stem (ES) cell by homologous recombination and produced heterozygous and homozygous mice using standard methods. An IFIX II genomic library from mouse strain 129/SvJ DNA (Stratagene) was screened with a genomic mclk1 fragment, and six overlapping genomic clones were obtained. Genomic DNA fragments from two clones were subcloned into Bluescript SK and characterized in detail. A 7 kb NotI-BamHI fragment containing part of the mclk1 promoter and exons 1, II and III was subcloned into Bluescript SK (pL5). A 1.6 kb fragment containing part of the exon 11 and the exon III was removed from pL5 by Stul/BamHI digestion and replaced with a neomycin cassette consisting of a 1.1 kb XhoI blunted-BamHI fragment from pMC1 Neo polyA to produce pL5+Neo. A 2.8 kb PstI-SacI genomic fragment containing introns IV and V and 500 bp from 5′UTR region was subcloned in Bluescript (pL15). A 2.5 kb EcORV-XhoI fragment from pL15 was inserted into the SmaI-XhoI sites of pL5+Neo to produce the final replacement targeting vector pL17. A KpnI fragment from the targeting vector was isolated and electroporated into R1 embryonic stem (ES). Successfully targeted clones were identified by Southern blot analysis. Genomic DNA was digested with BgIll, and then hybridized with a 3′external probe flanking the 3′ region of the targeting vector (SacI-XhoI fragment). A neomycin probe was used to detect random integrations in the genome. ES clones were injected into CD-1 mouse blastocysts and germline transmission was obtained. Out of 2000 G418-resistant clones analyzed, 4 were homologous recombinants. Two independently targeted ES cell clones with the correct karyotype were used to generate homozygous (−/−) mclk1 mice. FIGS. 2A, C and D display the maps of the wild-type mclk1 locus and of the targeting vector, where black boxes represent exons. The targeting vector consists of the replacement of a part of exon II and the exons III and IV by the neomycin gene, indicated as a white box in FIG. 2. The restriction enzymes sites indicated are: BamHI; B, BgIll; E, EcORI; K, KpnI; R, EcORV; S, SacI; X, XhoI. The genomic sequence of the Mus musculus wild-type mclk-1 locus and mutant knock-out allele of mclk-1 is given in FIGS. 6A-E (SEQ ID NO: 15) and 7A-E (SEQ ID NO: 16) respectively.

For genotype determinations, DNA was prepared from tails of aduit mice or yolk sacs of embryos. Southern blot analysis was done as described above. PCR was done for 30 cycles (95° C., 30 sec; 58° C., 30 sec; 72° C., 30 sec). The primers used to detect wild-type mclk1 allele were as follows: forward (KO5) 5′-ggt gaa gtc ttt tgg gtt tga gca t-3′ (SEQ ID NO: 17); reverse (KO6) 5′-tgt cta agg tca tcc ccg aac tgt g-3′ (SEQ ID NO: 18). They amplify a band of 302 bp. The targeted mclk1 allele was detected with the primers KO7 (5′-gcc agc gat atg act cag tgg gta a-3′) (SEQ ID NO: 19) and KO8 (5′-cac ctt aat atg cga agt gga cct g-3′) (SEQ ID No: 20), which give a product of 397 bp. FIG. 2E shows the PCR analyses.

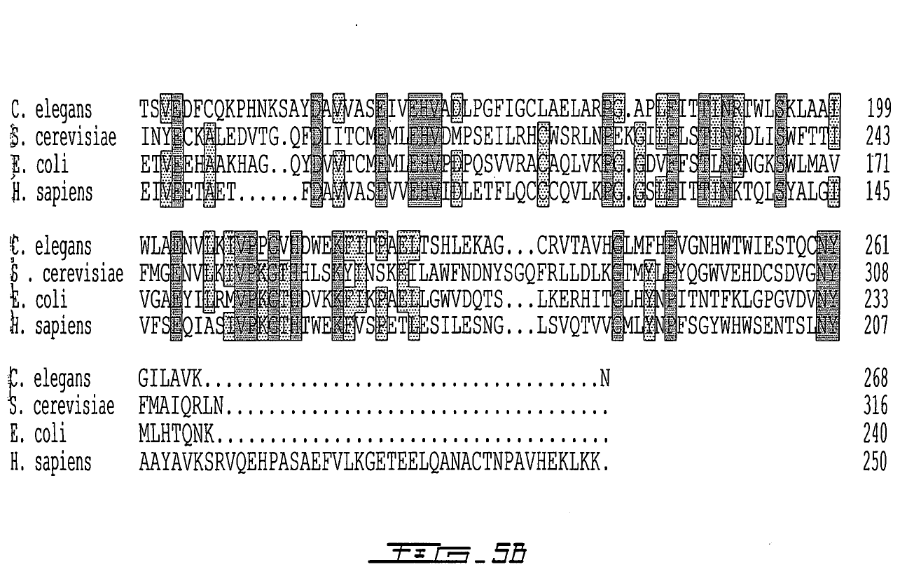

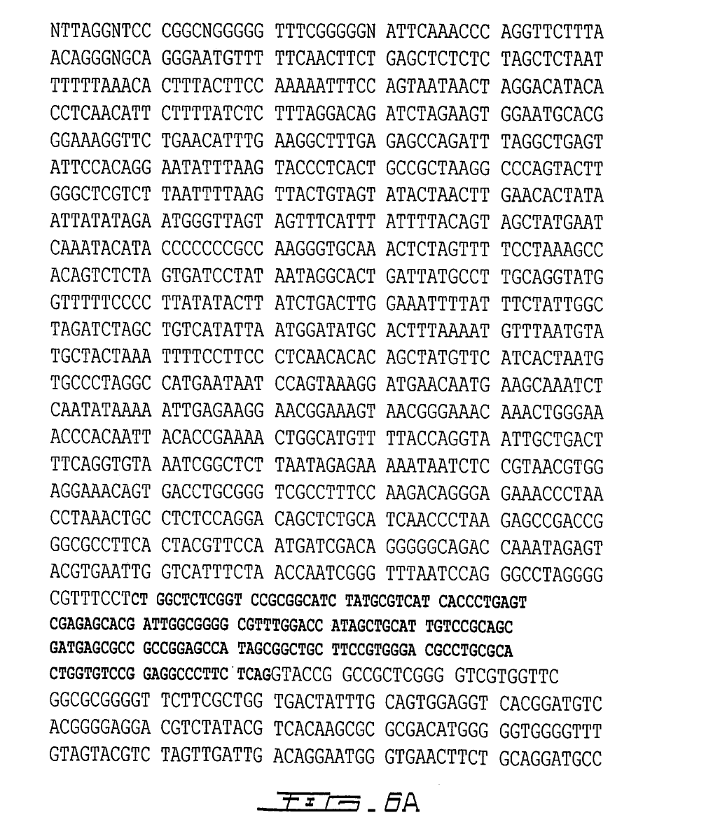

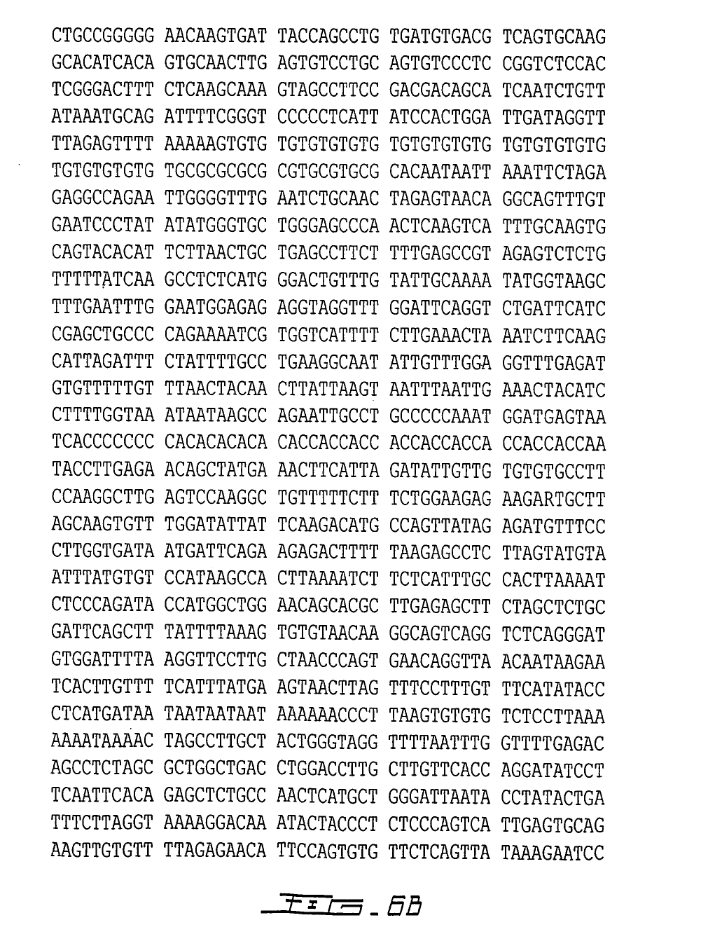

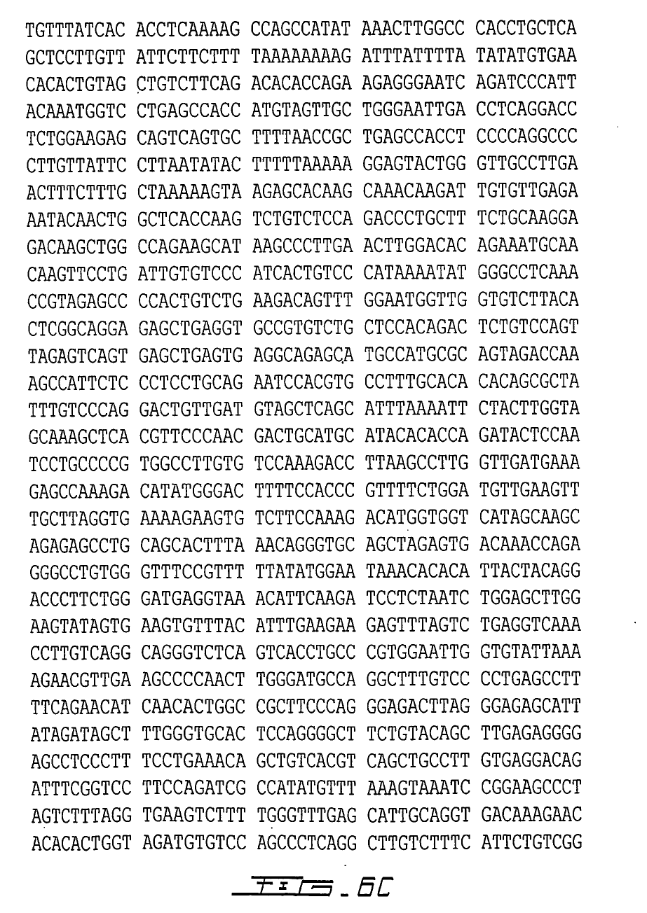

Heterozygous (+/−) mice are viable and fertile. They show no obvious anatomical or behavioral defects. However, after crossing heterozygous male and female mice, no new born (−/−) mice were observed in more than 81 offspring (Table 7), indicating that homozygous disruption of mclk1 results in embryonic lethality. To determine the nature of the lethality, embryos from heterozygous intercrosses were analyzed at different days of gestation (Table 7). mclk1 (−/−) embryos were present at expected mendelian frequencies at E8.5. By E13.5, however, all mclk1 (−/−) embryos detected were in the process of being resorbed. The homozygous embryos also showed a developmental delay that is clearly evident by day 9.5 post coitum (E9.5) (FIG. 3). The mutant is dramatically smaller compared to the wild-type littermate.

TABLE 7
Genotype distribution from mclk1 heterozygous crosses

| Stage | Total | +/+# | +/− | | −/− | | n.d. |
|---|---|---|---|---|---|---|---|
| E 8.5 | 74 | 18 (24%) | 40 | (54%) | 14 | (19%) | 2 |
| E 9.5 | 85 | 23 (27%) | 48 | (56%) | 12 | (14%) | 2 |
| E | 181 | 50 (28%) | 114 | (63%) | 16 | (9%) | 3 |
| 10.5 | | | | | | | |
| E | 137 | 35 (26%) | 84 + 2* | (63%) | 12 + 1* | (9%) | 2 + 1* |
| 11.5 | | | | | | | |
| E | 66 | 8 (12%) | 41 + 2* | (65%) | 2 + 2* | (6%) | 1 + 10* |
| 12.5 | | | | | | | |
| New- | 81 | 26 (32%) | 55 | (68%) | 0 | | — |
| born | | | | | | | |

n.d.: not determined.
*Embryos being resorbed.
#The genotype of embryos was determined by PCR analysis and that of pups by southern blotting, as described in Methods.

Northern blot analysis of total E11.5 embryo RNA showed that the amount of mclk1 mRNA was reduced by approximately 50% in heterozygous embryos when compared to normal embryo and could not be detected in mclk1 (−/−) embryos. FIG. 2B shows Northern blot analyses of total RNA levels in tissues from mclk1 +/+ and +/−mice and from E 11.5 mclk1 +/+, +/− and −/− littermates. The expression level of cox1, a mitochondrially encoded subunit of cytochrome oxidase (complex IV), is shown as one of the controls. The expression level of cox1 gives a good measure of the capacity for oxidative phosphorylation in a given tissue. Northern blots were performed using the full length mouse mclk1 cDNA as a probe. The decreased level observed in homozygous embryos is likely to be due to the beginning of the resorption process. An approximately 50% decrease of mclk1 transcript was observed in liver, heart, kidney, muscle, stomach and cerebellum of 44-day old mclk1 (+/−) mouse as compared to wild-type littermates. Immunoblotting with a polyclonal antibody revealed a band of ˜21 kDa in liver and heart extracts from (+/+) and (+/−) mice. This signal was reduced by 50% in (+/−) mice as compared to the (+/+) mice (FIG. 2D). The results confirmed that the mclk1 mutation is a null mutation and demonstrated a gene-dosage effect of reduced protein levels in (+/−) mice. Total protein extracts from liver and heart of two day-old mice were probed with antibodies against mCLKI and against the controls COX1 and Porin. Porin is a protein of the outer mitochondrial membrane encoded in the nucleus. Western blots were performed using monoclonal antibodies against cytochrome oxidase subunits I (1D6-E1-A8) and IV (20E8-C12) from Molecular Probes, and a monoclonal antibody against human porin 31 HL was from Calbiochem.

The amounts of ubiquinone-9 (UQ₉) and ˜10 (UQ₁₀) in homogenates of mclk1 (+/+), (+/−) and (+/−) embryos were determined by HPLC. Cell-free extracts for quinone analysis and enzyme activity measurements were prepared as follows. The samples were homogenized in 50 mM potassium phosphate buffer (pH 7.4), and centrifuged at 1,000×g for 5 min at 4° C. The supernatants were used for the determination of quinone content and the measurements of enzyme activity. Protein concentration was determined with bovine serum albumin as the standard. Quinones were extracted as described (Miyadera, H. et al., (2001). J Biol Chem 276, 7713-6), with slight modifications. Briefly, the quinones extracted in n-hexane/EtOH were dried under nitrogen gas, dissolved in acetone, and left at −80° C. After 30 minutes, the samples were centrifuged at 17,000×g, 15 min, 4° C., and the supernatant was dried under nitrogen gas. The residue was dissolved in EtOH, vortexed for 2 min, and applied to an HPLC (Model 100A, Beckman) equipped with a guard column, and an analytical column (CSC 80 \dot{a}, ODS2, C-18, 5 μm, 4.6×250 mm). The mobile phase was methanol/ethanol (70/30, v/v) with a flow rate of 2 m/min. The elution was monitored by a wavelength detector (165 variable wavelength detector, Beckman) at 275 nm. The concentration of quinones was determined spectrophotometrically as described (Miyadera, H. et al., (2001). J Biol Chem 276, 7713-6).

For mclk1 +/+embryos, a major peak elutes at 11.9 minutes and is identical to standard UQ for elution time. A smaller peak around 17.3 minutes corresponds to UQ₁₀. The quinone profile of heterozygous mclk1(+/−) embryos is identical to that of the wild type. The amount of UQ₉ and UQ₁₀ were similar in wild-type and heterozygous embryos (Table 8).

However, the presence of neither UQ₉ nor UQ₁₀ was observed in mclk1 (−/−) embryos (Table 8). These mutant embryos instead exhibited a major peak eluting 0.46 minutes earlier than UQ₉, which fits the criteria for being DMQ₉.

TABLE 8
Quinone content of ES cells and embryos

| | | Quinone type | | |
|---|---|---|---|---|
| | | DMQ₉ | UQ₉ | UQ₁₀ |
| Sample | Genotype | (ng/mg protein) | (ng/mg protein) | (ng/mg protein) |
| Embryos | +/+ | ND | 126.7 | 13.6 |
| | +/− | ND | 125.8 | 14.5 |
| | −/− | 37.1 | ND | ND |
| ES cells | ES1 (+/+) | ND | 265 | 16.8 |
| | ES2 (+/−) | ND | 89.5 | 4.2 |
| | ES7 (−/−) | 38.4 | ND | ND |

N.D.: not detected.
mclk1 (+/+), (+/−) and (−/−) ES cell lines were derived from E3.5 blastocysts obtained from heterozygous matings as per standard procedures. The quinone profiles observed in these lines follow the same pattern as those obtained from the equivalent mutant embryos, including concentration (Table 4). In particular, only DMQ₉ was detected in the mclk1 (−/−) ES cell line (ES 7).

As in the case of the clk-1 mutants in C. elegans, the DMQ produced in mclk1 mutants appears to be sufficient for the maintenance of a relatively high level of oxygen consumption (62% of the wild type). It is surprising that such levels of mitochondrial function are insufficient to carry out embryogenesis. However, a number of elements could participate in the severity of the phenotype. Again, UQ is found in almost all biological membranes and is known to be a co-factor of the uncoupling proteins (UCP) in the mitochondria, to regulate the permeability transition pore, and to function in plasma membrane and lysosomal oxido-reductase systems. Although DMQ can partially replace UQ in the respiratory chain, it is possible that DMQ is less efficient as a UQ analogue for some of the other functions of UQ, whose resulting impairement participates in the severity of the phenotype. Finally, it has recently been discovered that, in bacteria, quinones are the primary signal for the regulation of growth in response to oxygen availability. Given the conservation between prokaryotes and eukaryotes of crucial molecular mechanisms that sense environmental signals (e.g. the PAS domain proteins), the full UQ deficiency of mclk1 mutants directly affects the regulation of embryonic growth.

Studies of Tissue-Specific and Temporally Controlled Knockout of the mclk1 Gene

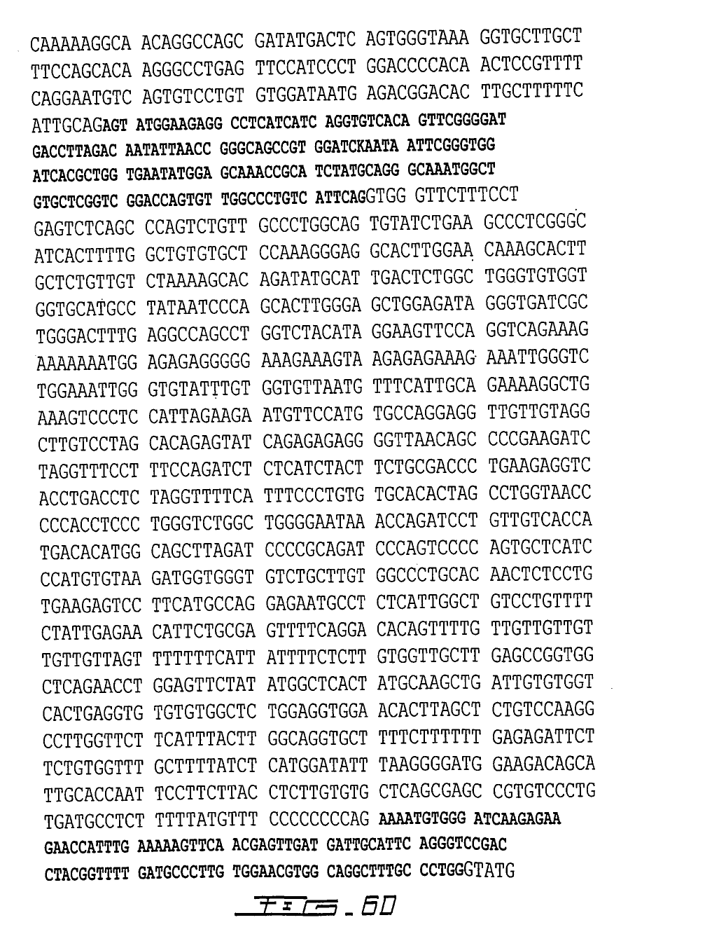

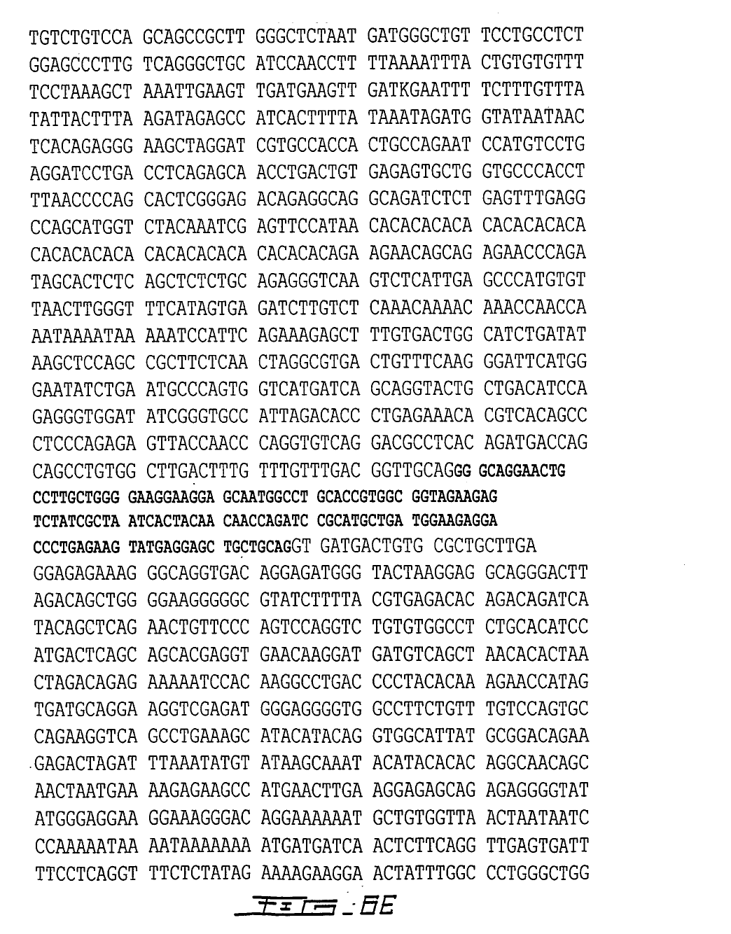

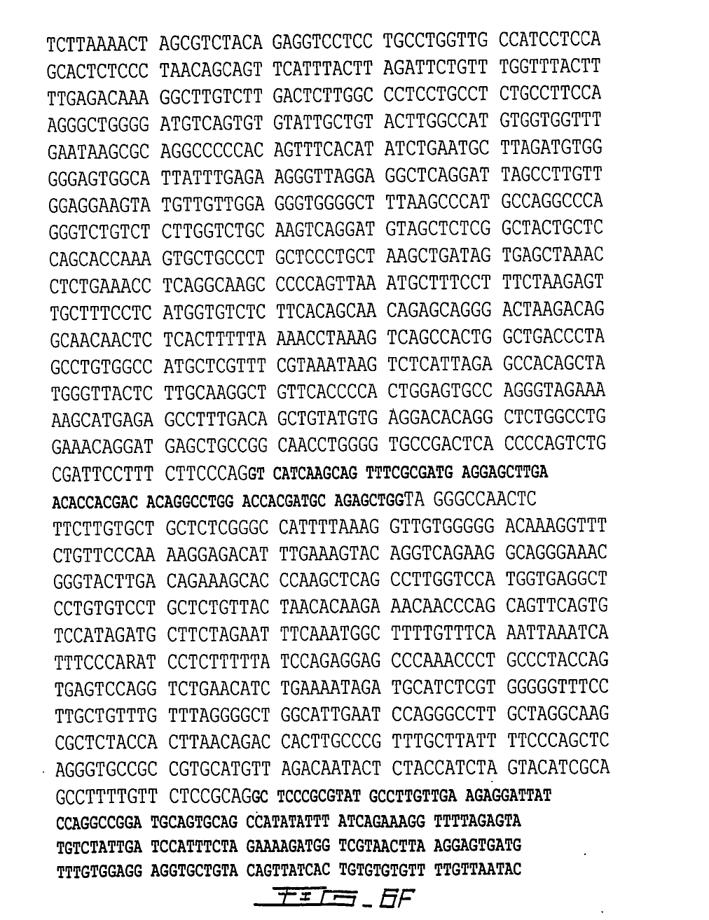

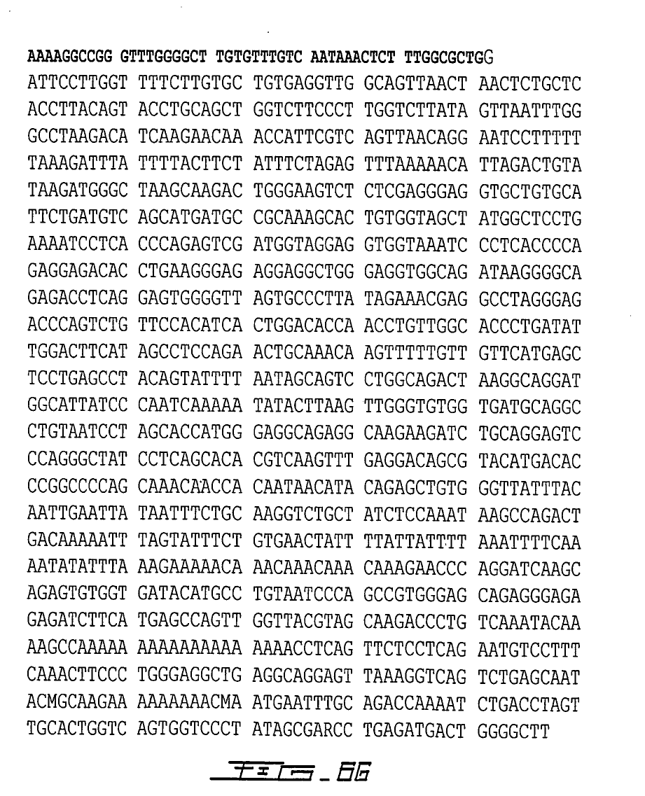

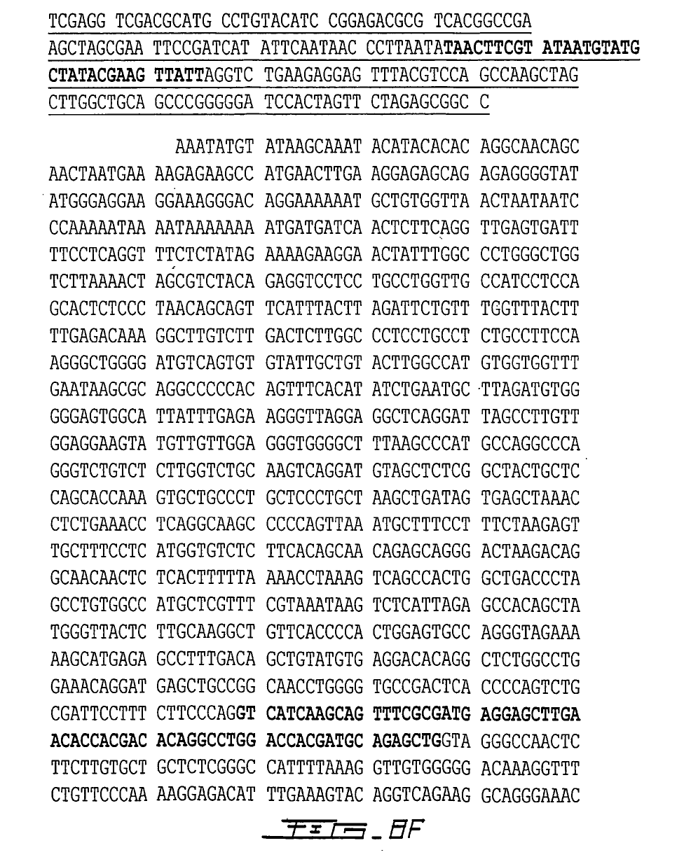

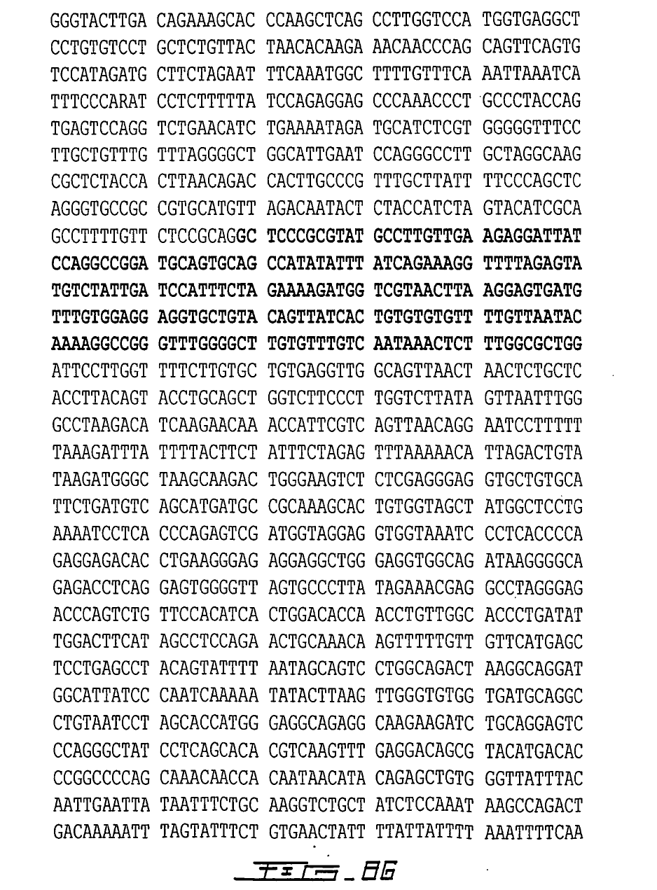

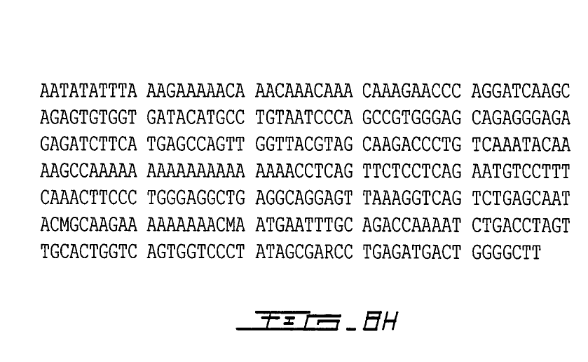

In addition, studies of tissue-specific and temporally controlled knockout of mclk1 gene have been initiated in Mus musculus. mclk1^{flox} allele was created and chimeric mouse was generated as follows. In order to investigate the functional role of mCLK1 protein in specific cells, the technique of conditional gene inactivation was used with Cre-loxP mediated recombination. To produce an mclk1 allele that can be modified by Cre-recombination, a targeting vector containing approximately 7.5 kb of mclk1 genomic DNA was constructed in which a selection cassette flanked by loxP sites was introduced downstream of exon 4 with a third loxP site upstream of exon 2 (see FIGS. 4A-C and FIGS. 8A-E (SEQ ID NO:21)). In FIGS. 4A-C, a horizontal line represents clk1 genomic DNA. Exons are represented by unfilled boxes. The gray box represents a neo-TK expression cassette, with the direction of neo and TK transcription indicated by arrows. The black head arrows represent loxP sites. The restriction sites are: BglII (B), Bspel (P), EcORI (E), HindIII (H), SacI (S), Swal (W), XhoI (X). Following transfection of ES cells, homologous recombinants were identified by Southern blot analysis. Genomic DNA was digested with BglII, and then hybridized with the 3′external probe flanking 3′region of the targeting vector (SacI-XhoI fragment). After analysing the promising neomycin-resistant clones by extensive southern blot, three clones (30, 48 and 84) showed the correct homologous recombination (FIG. 4). FIG. 4A displays a schematic representation of mclk1 locus and the targeting vector. The different probes used for southern blot are drawn. FIG. 4B gives the expected fragment sizes upon digestion with the different enzymes. FIG. 4C displays the southern blot were performed on BgIll or EcORI digested DNA using different probes. A 9 kb band obtained if there is insertion of the selection cassette flanked by loxP sites downstream of exon 4 without insertion of the third loxP site upstream of exon 2, and is indicated by a * in FIG. 4C.

A detailed description of the generation of the mclk1^{flox} allele follows. mclk1 genomic DNA was isolated from a strain 129/SvJ mouse library (Stratagene) and a HindIII-XhoI fragment of approximately 7.5 kb containing exons 2, 3, 4, 5 and 6 was subcloned into pBluescript. A primer containing a loxP site (3′-CCG GAG CTA GCG AGC TCG GM TM CTT CGT ATA ATG TAT GCT ATA CGA AGT TAT GGC GAA TT-5′) (SEQ ID NO: 11) was introduced into a Bsepi site upstream the exon 2. A cassette containing the neor and HSV-tk genes flanked by two loxP sites was inserted into the SwaI site in intron 4 to yield the targeting replacement vector pL75. This cassette, a 4.3 kb Xhol/Not I fragment, was isolated from the plasmid CDLNTKL (SEQ ID No: 12) and the recessed 3′ termini were filled with Klenow enzyme.

To generate homologous recombinants, R1 ES cells derived from 129/Sv mice (at passage 12) were electroporated with HindIII-XhoI targeting vector fragment. Homologous recombinants were identified by Southern blot hybridization. Genomic DNA was digested with BglII, and then hybridized with the 3′external probe flanking the 3′region of the targeting vector (SacI-XhoI fragment). Other probes were used to detect random insertions in the genome. Hybridizations were performed for 16 hours at 65° C. in 6×SSC, 5× Denhart, 0.5% SDS. Blots were then washed for 20 min each, twice 3×SSC, 0.1% SDS, then twice with 1×SSC, 0.1% SDS.

To generate type I and type II deletions, 5×106 homologous recombinant cells were electroporated with 25 μg pBS185 containing the cre-recombinase gene, plated and selected 48 h later with 2 μM gancyclovir. Surviving clones were analyzed by Southern blot. Genomic DNA was digested with SacI, and then hybridized with the 3′external probe flanking 3′region of the targeting vector (SacIXhoI fragment).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1-25. (Canceled)

26. A mouse comprising a knock-out of a murine clk-1 gene, wherein said mouse exhibits an increased life span relative to a wild-type mouse.

27 (Canceled)

28. A mouse cell comprising a knock-out of a murine clk-1 gene.

29-41. (Canceled)

42. The mouse of claim 26, wherein a plurality of cells of said mouse comprise said knock-out.

43. The mouse of claim 26, wherein all somatic cells of said mouse comprise said knock-out.

44. The mouse of claim 43, wherein the amount of murine clk-1 mRNA is about 50% less relative to a wild-type mouse.

45. The mouse of claim 26, which exhibits altered cellular metabolism, altered development rate, altered behavioral rate, or altered cell cycle.

46. The mouse of claim 45, which exhibits a decrease in cellular metabolism or a decrease in developmental rate, relative to a wild-type mouse.

47. A mouse embryo comprising a plurality of cells which comprise a knock-out of a murine clk-1 gene.

48. The mouse embryo of claim 47, wherein all somatic cells of said mouse embryo comprise a knock-out of a murine clk-1 gene.

49. The mouse embryo of claim 47, wherein said plurality of cells comprise a knock-out of a murine clk-1 gene at both alleles.

50. The mouse embryo of claim 47, wherein all somatic cells of said mouse embryo comprise a knock-out of a murine clk-1 gene at both alleles.

51. The mouse embryo of claim 48, wherein the amount of mouse clk-1 mRNA is about 50% less relative to a wild-type mouse embryo.

52. The mouse embryo of claim 49, which exhibits a decrease in ubiquinone level relative to a wild-type mouse embryo.

53. The mouse embryo of claim 49, which exhibits an increase in demethoxyubiquinone level relative to a wild-type mouse embryo.

54. The mouse cell of claim 28, which is heterozygous in the mouse clk-1 locus.

55. The mouse cell of claim 28, which is homozygous in the mouse clk-1 locus.

56. The mouse cell of claim 54, wherein the amount of mouse clk-1 mRNA is about 50% less relative to a wild-type mouse embryonic stem cell.

57. The mouse cell of claim 28, which exhibits a decrease in ubiquinone level relative to a wild-type mouse embryonic stem cell.

58. The mouse cell of claim 55, which exhibits an increase in demethoxyubiquinone level relative to a wild-type mouse embryo.

59. The mouse cell of claim 54 or 55, wherein the knock-out of the murine clk-1 gene is conditional.

60. The mouse cell of claim 59, wherein the knock-out is mediated by Cre-loxP recombination.

61. The mouse embryo of claim 48 or 49, wherein the knock-out of the murine clk-1 genesis conditional.

62. The mouse embryo of claim 61, wherein the knock-out is mediated by Cre-loxP recombination.

63. The mouse of claim 42 or 43, wherein the knock-out of the murine clk-1 gene is conditional.

64. The mouse of claim 63, wherein the knock-out is mediated by Cre-loxP recombination.

65. The mouse embryo of claim 61, wherein the knock-out of the murine clk-1 gene is tissue-specific or organ-specific.

66- The mouse embryo of claim 62, wherein the knock-out of the murine clk-1 gene is tissue-specific or organ-specific.

67. The mouse embryo of claim 61, wherein the knock-out of the murine clk-1 gene is temporally controlled.

68. The mouse embryo of claim 62, wherein the knock-out of the murine clk-1 gene is temporally controlled.

69. The mouse of claim 63, wherein the knock-out of the murine clk-1 gene is tissue-specific or organ-specific.

70. The mouse of claim 64, wherein the knock-out of the murine clk-1 gene is tissue-specific or organ-specific.

71. The mouse of claim 63, wherein the knock-out of the murine clk-1 gene is temporally controlled.

72. The mouse of claim 64, wherein the knock-out of the murine clk-1 gene is temporally controlled.

73. The mouse cell of claim 28 which is from a cell line.

74. The mouse cell of claim 28 which is an embryonic stem cell.

75. The mouse cell of claim 28 which is derived from liver, heart, kidney, muscle, stomach, or cerebellum tissue.
